(19) United States
(12) Patent Application Publication
(10) Pub. No.: US 2005/0059664 A1
(43) Pub. Date: Mar. 17, 2005

(54) Novel methods for identifying improved, non-sedating alpha-2 agonists

(75) Inventors: Daniel W. Gil, Corona Del Mar, CA (US); John E. Donello, Dana Point, CA (US)

Correspondence Address: Carlos A. Fisher;ALLERGAN, INC.-T2-7H, 2525 Dupont Drive, Irvine, CA, 92612, US

(73) Assignee: Allergan, Inc.

(21) Appl. No.: 10/891,740

(22) Filed: Jul. 15, 2004

Related U.S. Application Data
(60) Provisional application No. 60/502,840, filed on Sep. 12, 2003.

(51) Int. Cl. A61K 31/498

(52) U.S. Cl. 514/249

(57) ABSTRACT

The present invention provides methods of preventing or alleviating sympathetically-enhanced conditions, neurological conditions, ocular conditions and chronic pain without concomitant sedation by peripherally administering to a subject an effective amount of an α-2A/α-1A selective agonist, thereby preventing or alleviating the condition or chronic pain without concomitant sedation, where the selective agonist has an α-1A efficacy less than that of brimonidine or a ratio of α-1A/α-2A potency greater than that of brimonidine.

This patent application claims benefit of priority under 35 USC § 119(e) to provisional patent application No. 60/502,840, filed Sep. 12, 2003, which is hereby incorporated by reference herein.

### BACKGROUND OF THE INVENTION

1. Field of the Invention

The invention relates generally to molecular medicine and, more particularly, to α-2 adrenergic agonists that are highly selective for an α-2 adrenergic receptor as compared to an α-1 adrenergic receptor.

2. Background Information

A variety of conditions can be mediated, at least in part, by the sympathetic nervous system including a variety of conditions associated with stress. Such sympathetically-enhanced conditions include, without limitation, sensory hypersensitivity, for example, sensory hypersensitivity associated with fibromyalgia or headache such as migraine; gastrointestinal diseases such as irritable bowel syndrome and dyspepsia; dermatological conditions such as psoriasis; cardiovascular disorders; tachycardias; disorders of peripheral vasoconstriction such as Raynaud's Syndrome and scleroderma; panic attack; metabolic disorders such as type II diabetes, insulin-resistance and obesity; disorders of muscle contraction including disorders of skeletal muscle contraction, disorders of smooth muscle contraction, spasticity, and disorders of muscle contraction associated with tension-type headache; behavioral disorders such as over-eating and drug dependence; and sexual dysfunction.

Unfortunately, treatment of sympathetically-enhanced conditions with α-2 agonists can be unsatisfactory due to concomitant sedative effects. This same problem limits effective α-2 adrenergic agonist treatment of other conditions including neurological conditions, ocular conditions and chronic pain. Thus, there is a need for novel methods of preventing or alleviating sympathetically-enhanced conditions, neurological conditions, ocular conditions and chronic pain without concomitant sedation and for convenient screening methods of identifying effective, non-sedating α-2 agonists for use as therapeutics. The present invention satisfies these needs and provides related advantages as well.

### SUMMARY OF THE INVENTION

The present invention provides a method of preventing or alleviating a sympathetically-enhanced condition without concomitant sedation by peripherally administering to a subject an effective amount of an α-2A/α-1A selective agonist, thereby preventing or alleviating the sympathetically-enhanced condition without concomitant sedation, where the selective agonist has an α-1A efficacy less than that of brimonidine or a ratio of α-1A/α-2A potency greater than that of brimonidine.

Further provided herein is a method of preventing or alleviating chronic pain without concomitant sedation by peripherally administering to a subject an effective amount of an α-2A/α-1A selective agonist, thereby preventing or alleviating the chronic pain without concomitant sedation, where the selective agonist has an α-1A efficacy less than that of brimonidine or a ratio of α-1A/α-2A potency greater than that of brimonidine.

The present invention additionally provides a method of preventing or alleviating a neurological condition without concomitant sedation by peripherally administering to a subject an effective amount of an α-2A/α-1A selective agonist, thereby preventing or alleviating the neurological condition without concomitant sedation, where the selective agonist has an α-1A efficacy less than that of brimonidine or a ratio of α-1A/α-2A potency greater than that of brimonidine.

Also provided herein is a method of preventing or alleviating an ocular condition without concomitant sedation by peripherally administering to a subject an effective amount of an α-2A/α-1A selective agonist, thereby preventing or alleviating the ocular condition without concomitant sedation, where the selective agonist has an α-1A efficacy less than that of brimonidine or a ratio of α-1A/α-2A potency greater than that of brimonidine.

The present invention further provides a method of screening for an α-2A/α-1A selective agonist that prevents or alleviates sympathetically-enhanced conditions without concomitant sedation upon peripheral administration. Such a screening method is practiced by determining the functional selectivity of an agent for activating an α-2A receptor as compared to an α-1A receptor, where an agent which is highly selective for activating an α-2A receptor as compared to an α-1A receptor is an α-2A/α-1A selective agonist that prevents or alleviates sympathetically-enhanced conditions without concomitant sedation upon peripheral administration.

### BRIEF DESCRIPTION OF THE DRAWINGS

FIG. 1 shows the tactile hypersensitivity observed with several distinct chemical models. Each experimental group included 5-6 wildtype mice. Tactile hypersensitivity was assessed as described below; sensitization scores determined every 5 minutes during the 35 minute measurement period were summed and calculated as the mean+/−SEM. Each group was compared to a vehicle control using an unpaired two-tailed t-test (* p<0.01, ** p<0.001). (a) Spinal injection of the α-1 agonist, phenylephrine, induces tactile hypersensitivity in a dose dependent fashion. Phenylephrine (filled circle) was injected intrathecally at various doses. The α-1 antagonist, 5-MU (30 ug/kg i.p.; filled square) was administered 15 minutes prior to intrathecal administration of 30 ng phenylephrine. (b) Systemic phenylephrine induces tactile hypersensitivity in a dose dependent fashion. Phenylephrine (filled circle) was injected intraperitoneally at various doses. The α-1 antagonist, 5-MU (30 ug/kg i.p.; filled square) was administered 15 minutes prior to administration of 30 ng/kg phenylephrine. (c) Spinal sulprostone, a selective EP₁/EP₃ agonist, induces chemical tactile hypersensitivity in a dose responsive fashion. Increasing doses of sulprostone (filled circle) were injected intrathecally. An EP₁ antagonist (100 ng i.t.; filled square) was injected 15 minutes prior to administration of 200 ng sulprostone. (d) Spinal administration of NMDA induces tactile hypersensitivity in a dose responsive fashion. NMDA (filled circle) was injected intrathecally at various doses. The NMDA antagonist, memantine (1 ug i.t.; (filled square), was injected 15 minutes prior to administration of 100 ng NMDA.

FIG. 2 shows that the increased sympathetic tone of α-2A and α-2C knockout mice enhances induction of tactile hypersensitivity by α-1 receptor activation. Wildtype (filled circle), α-2A knockout (filled square), and α-2C knockout (filled triangle) mice were injected intraperitoneally with increasing doses of phenylephrine and assayed for tactile hypersensitivity. α-2A knockout mice were pretreated with 50 mg/kg i.p. guanethidine to cause a temporary chemical sympathectomy 24-30 hours prior to an i.p. injection with phenylephrine (open square). Each group of mice consisted of 5-6 animals. The mean sensitization score and SEM were calculated and compared to a vehicle control group using an unpaired two-tailed t-test (* p<0.01, ** p<0.001).

FIG. 3 shows that the sympathetic nervous system enhances sulprostone-induced tactile hypersensitivity. Wildtype (filled circle), α-2A (filled square), and α-2C (filled triangle) knockout mice were injected intrathecally with increasing doses of sulprostone and assayed for tactile hypersensitivity. α-2A knockout mice were pretreated with guanethidine (50 mg/kg i.p.) to cause a temporary chemical sympathectomy 24 hours prior to an intrathecal sulprostone injection (open square). Each group of mice consisted of 5-6 animals. The mean sensitization score and SEM were calculated and compared to a vehicle control group using an unpaired two-tailed t-test (* p<0.01, ** p<0.001).

FIG. 4 shows that α-2 knockout mice do not exhibit altered NMDA-induced tactile hypersensitivity. Wildtype (filled circle), α-2A (filled square), and α-2C (filled triangle) knockout mice were injected intrathecally with increasing doses of NMDA. Each group of 5-6 mice was scored for tactile hypersensitivity. The mean response and SEM were calculated and compared to a vehicle control group using an unpaired two-tailed t-test (* p<0.01, ** p<0.001).

FIG. 5 shows that α-adrenergic agonists differ in alleviation of sympathetically-enhanced sensory hypersensitivity. The response of 5-6 mice per group was scored; the mean response and SEM were calculated as described above. Each drug-treated group was compared to a vehicle control group using an unpaired two-tailed t-test (* p<0.01, ** p<0.001). (a) Spinal brimonidine and clonidine alleviate NMDA-induced tactile hypersensitivity in wildtype mice. Mice were injected intrathecally with DMSO vehicle or co-injected intrathecally with 100 ng NMDA and saline, 0.4 μg brimonidine (UK14304) or 1 μg clonidine. (b) Spinal brimonidine and clonidine alleviate sulprostone-induced tactile hypersensitivity in wildtype mice. Mice were injected intrathecally with DMSO vehicle or co-injected intrathecally with 200 ng sulprostone and saline, 0.4 μg brimonidine (UK14304) or 0.4 μg clonidine. (c) Spinal brimonidine and clonidine alleviate NMDA-induced tactile hypersensitivity in the α-2C knockout mice, but not in the α-2A knockout mice. Mice were injected intrathecally with DMSO vehicle or coinjected intrathecally with 100 ng NMDA and saline, 0.4 μg brimonidine (UK14304) or 1 μg clonidine. (d) Spinal brimonidine and clonidine differ in their ability to alleviate sulprostone-induced tactile hypersensitivity in the α-2C knockout mice. Mice were injected with DMSO vehicle or co-injected intrathecally with 200 ng (α-2C knockout) or 30 ng (α-2A knockout) sulprostone and saline, 0.4 μg brimonidine (UK14304) or 0.4 μg clonidine. α-2 agonist analgesia is absent in the α-2A knockout mice; clonidine analgesia is also lost in the α-2C knockout mice.

FIG. 6 shows that brimonidine, but not clonidine or tizanidine, alleviates sulprostone-induced tactile hypersensitivity in the absence of sedation. The dose-responsive anti-hypersensitive and sedative effects of three α-2 agonists (tizanidine, triangle; clonidine, square; and brimonidine, circle) were compared in models of sulprostone-induced tactile hypersensitivity and locomotor activity, respectively. The mean total sensitivity score and standard error of the mean was calculated and indicated as a solid line (left axis). Locomotor activity relative to vehicle-treated animals was expressed as a percentage, and the percent sedation calculated as 100% minus the percent locomotor activity and indicated as a hatched line (right axis).

FIG. 7 shows variable α-2 vs. α-1 agonist selectivity in α-adrenergic agonists clonidine and brimonidine. Increasing concentrations of phenylephrine (filled square), clonidine (filled diamond), tizanidine (filled circle), dexmeditomidine (filled triangle) and brimonidine (filled inverted triangle) were tested for α-1 and α-2 agonist activity using in vitro cell-based functional assays. (a, b) α-1A and α-1B agonist activity of α-adrenergic agonists. The increase in intracellular calcium in HEK cells stably expressing the bovine α-1A receptor (a) or the hamster α-1B receptor (b) following addition of various concentrations of α-adrenergic agonists was determined by measuring the change in fluorescence of a calcium-sensitive dye. Agonists were tested 6-15 times in triplicate, and the mean fluorescence and SEM calculated at each concentration. Results from a typical experiment are shown. (c, d) α-2A and α-2C agonist activity of α-adrenergic agonists. Inhibition of forskolin-induced cAMP accumulation in PC12 cells stably expressing the human α-2A receptor (c) or the human α-2C receptor (d) following addition of various concentrations of α-adrenergic agonists. Agonists were tested 3-5 times in triplicate, and the mean % inhibition and SEM calculated at each concentration. Results from a typical experiment are shown. (e) Co-administration of prazosin with clonidine restores clonidine-mediated analgesia in α-2C knockout mice. Wildtype (open bars) and α-2C knockout (hatched bars) mice were injected with vehicle, prazosin (100 ng/kg i.p.), sulprostone (200 ng i.t.), clonidine (400 ng i.t.) or various combinations as indicated. The tactile hypersensitivity of 5-6 mice per group was scored, and the mean response and SEM was calculated. Each drug-treated group was compared to a vehicle control group using an unpaired two-tailed t-test (* p<0.01, ** p<0.001).

FIG. 8 shows that Compound 1 is superior to brimonidine in its ability to alleviate sulprostone-induced tactile hypersensitivity in the absence of sedation. The dose-responsive anti-hypersensitive and sedative effects of four α-2 agonists were compared in models of sulprostone-induced tactile hypersensitivity and locomotor activity. Upper left panel: I.P. Brimonidine. Upper right panel: I.P. Dexmeditomidine. Lower left panel: Oral Compound 1. Lower right panel: I.P. Compound 2. The mean total sensitivity score and standard error of the mean were calculated (see solid line and solid symbols, left axis). Locomotor activity relative to vehicle-treated animals was expressed as a percentage, and the percent sedation calculated as 100% minus the percent locomotor activity (see hatched line and open symbols, right axis).

### DETAILED DESCRIPTION OF THE INVENTION

Adrenergic receptors mediate physiological responses to the catecholamines, norephinephrine and epinephrine, and are members of the superfamily of G protein-coupled receptors having seven transmembrane domains. These receptors, which are divided pharmacologically into α-1, α-2 and β-adrenergic receptor types, are involved in diverse physiological functions including functions of the cardiovascular and central nervous systems. The α-adrenergic receptors mediate excitatory and inhibitory functions: α-1 adrenergic receptors are typically excitatory post-synaptic receptors which generally mediate responses in an effector organ, while α-2 adrenergic receptors are located postsynaptically as well as presynaptically, where they inhibit release of neurotransmitters. Agonists of α-2 adrenergic receptors currently are used clinically in the treatment of hypertension, glaucoma, spasticity, and attention-deficit disorder, in the suppression of opiate withdrawal, as adjuncts to general anesthesia and in the treatment of cancer pain.

α-2 adrenergic receptors are presently classified into three subtypes based on their pharmacological and molecular characterization: α-2A/D (α-2A in human and α-2D in rat); α-2B; and α-2C (Bylund et al., Pharmacol. Rev. 46:121-136 (1994); and Hein and Kobilka, Neuropharmacol. 34:357-366 (1995)). The α-2A and α-2B subtypes can regulate arterial contraction in some vascular beds, and the α-2A and α-2C subtypes mediate feedback inhibition of norepinephrine release from sympathetic nerve endings. The α-2A subtype also mediates many of the central effects of α-2 adrenergic agonists (Calzada and ArtiZano, Pharmacol. Res. 44: 195-208 (2001); Hein et al., Ann. NY Acad. Science 881:265-271 (1999); and Ruffolo (Ed.), α-Adrenoreceptors: Molecular Biology, Biochemistry and Pharmacology S. Karger Publisher's Inc. Farmington, Conn. (1991)).

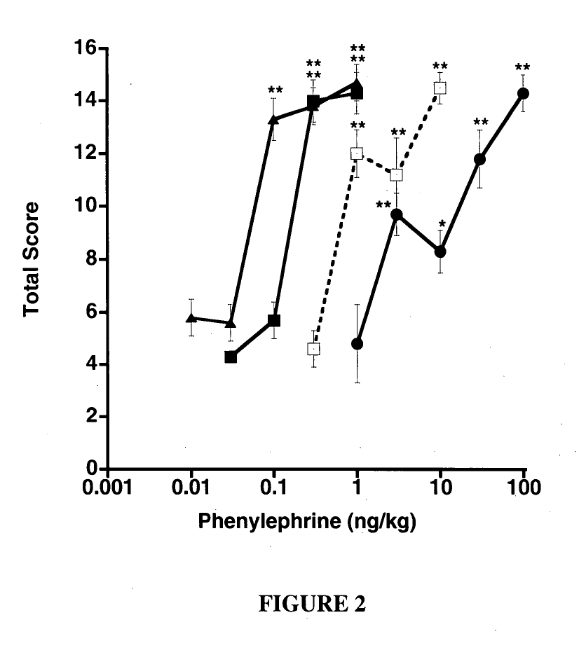

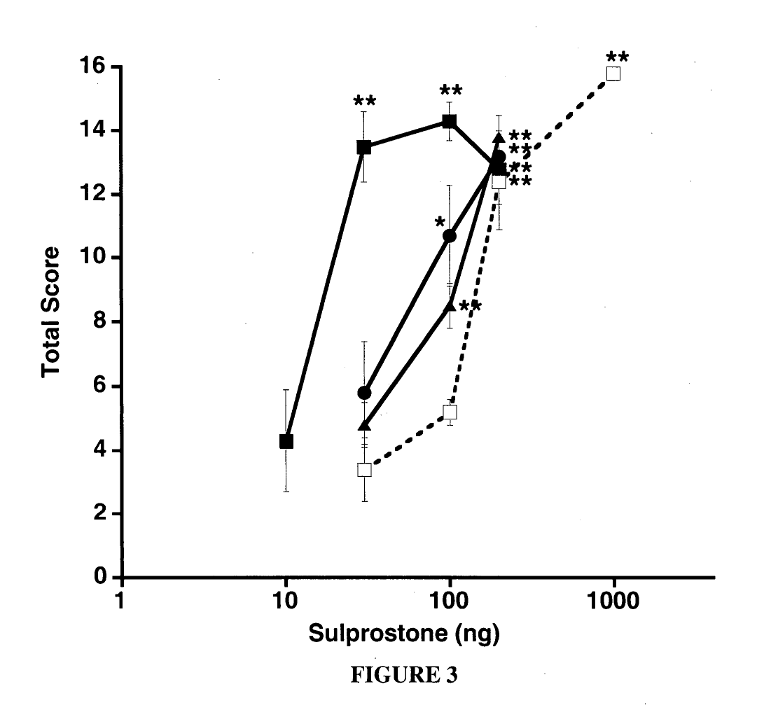

Previous studies have shown that norepinephrine has a higher affinity for the α-2C receptor (Kᵢ=650 nM) than the α-2A receptor (Kᵢ=5800 nM; Link et al., Mol. Pharm. 42:16-27 (1992)). Thus, the autoinhibitory action on norepinephrine release is mediated through the α-2C receptor at low concentrations of norepinephrine, and through the α-2A receptor at high concentrations of norepinephrine (Altman et al., Mol. Pharm. 56:154-161 (1999)). As a result, feedback inhibition of basal norepinephrine release is mediated by the α-2C receptor, while the α-2A receptor mediates feedback inhibition of release under conditions of high frequency stimulation (Hein et al., Ann. N.Y. Acad. Sci. 881:265-271 (1999)). As disclosed herein in Example I, the α-2C knockout mice, which have a decreased presynaptic inhibition of sympathetic outflow under basal (or low frequency stimulation) conditions, are more sensitive to augmentation of α-1 receptor activity through phenylephrine treatment (see FIG. 2). Furthermore, as shown herein in FIG. 3, α-2A knockout mice are more sensitive to sulprostone-induced tactile hypersensitivity, while in α-2C knockout mice, the sulprostone sensitivity is the same as that of wildtype mice. These results demonstrate that sulprostone treatment results in high frequency sympathetic nerve stimulation, as evidenced by the fact that only α-2A knockout mice, which lack presynaptic inhibition of high frequency sympathetic outflow, exhibit a decreased threshold to sulprostone-induced tactile hypersensitivity.

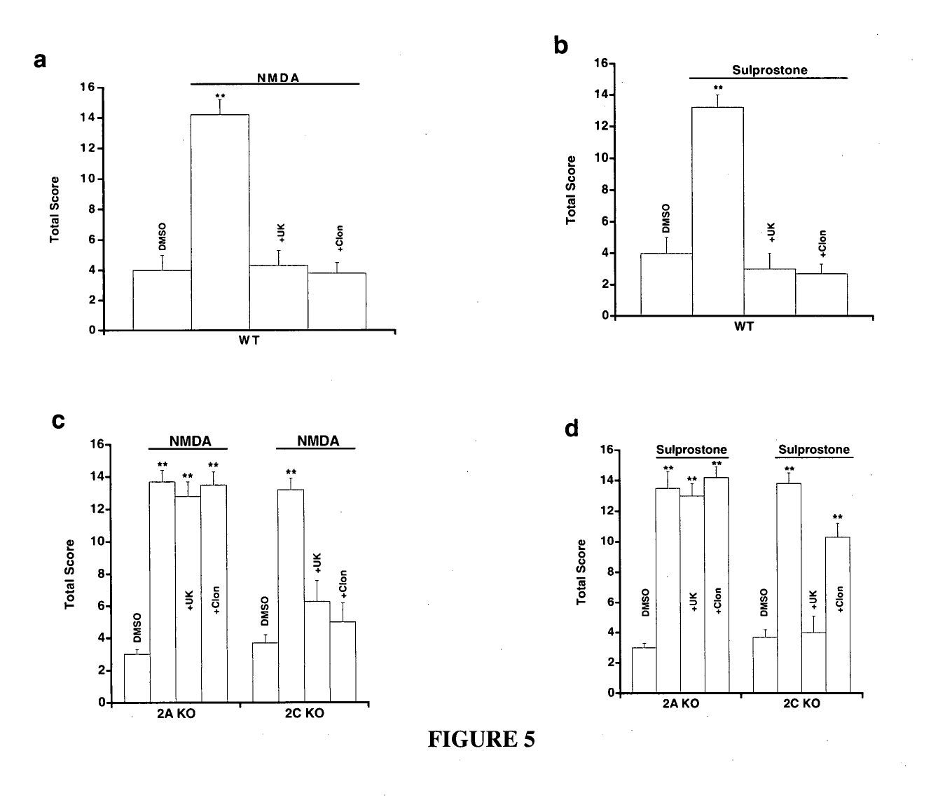

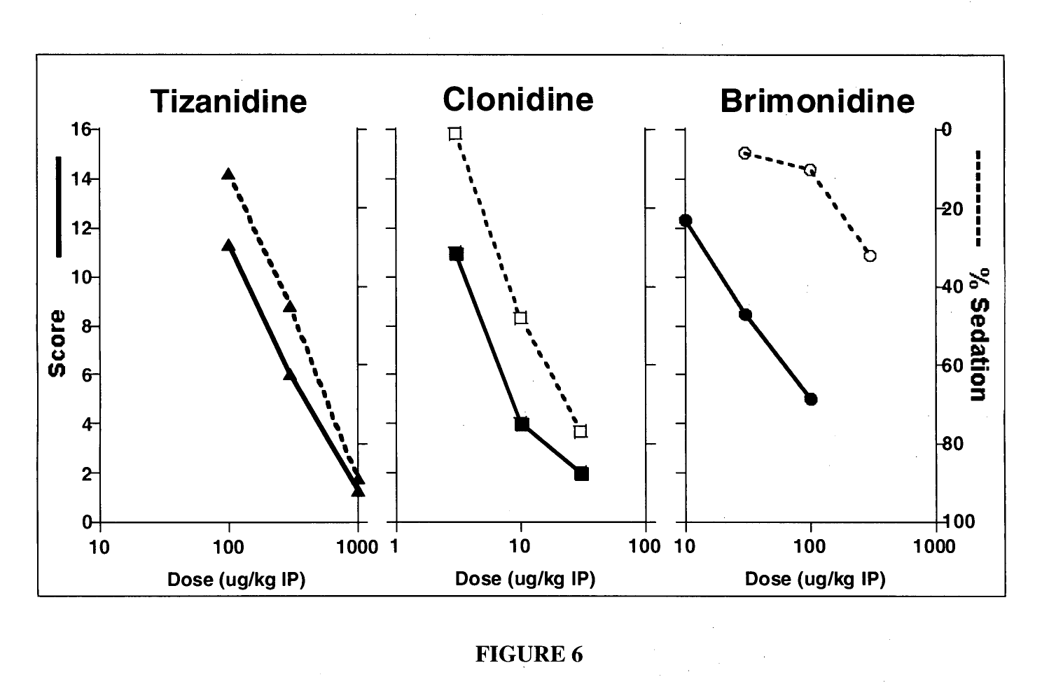

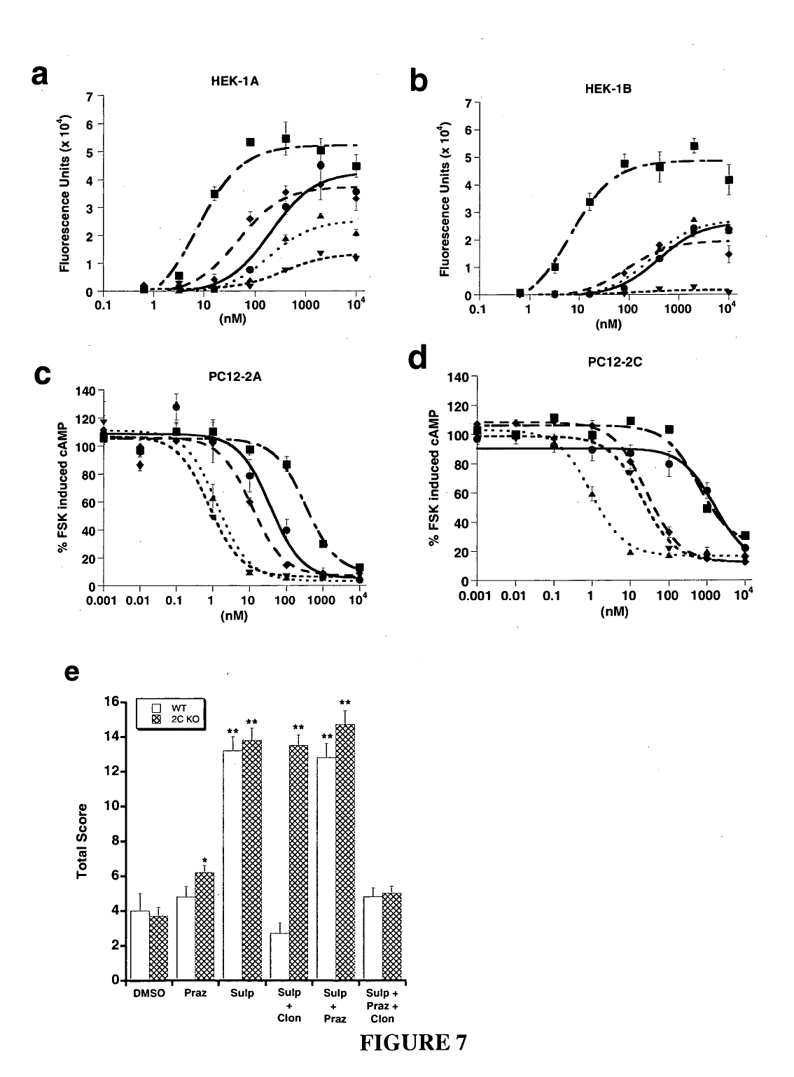

As disclosed herein in Example II, brimonidine was analgesic in both wild type and α-2C knockout mice with sulprostone-induced tactile hypersensitivity. In contrast, clonidine was analgesic in wild type mice but not in α-2C knockout mice (compare FIGS. 5b and d). As expected, neither clonidine nor brimonidine were analgesic in α-2A knockout mice, which lack the spinal α-2A adrenergic receptor which mediates analgesic activity. Thus, in α-2C knockout mice treated with sulprostone, which serve as a model for sympathetically-enhanced conditions, the pan-agonists brimonidine and clonidine have strikingly different activities. Additional results disclosed herein demonstrate that, in wild type mice, brimonidine, but not other pan-agonists such as tizanidine or clonidine, had analgesic activity without concomitant sedation (see FIG. 6). Furthermore, brimonidine was more selective (more than 1000-fold) for α-2 adrenergic receptors relative to α-1 receptors in functional assays as compared to other pan-agonists such as clonidine and tizanidine, which exhibited less than 10-fold selectivity (see FIG. 7 and Table 3). These results demonstrate the differential functional activity of the pan-agonists brimonidine and clonidine and indicate that α-2 versus α-1 functional selectivity can be advantageous in treating sympathetically-enhanced and other conditions without concomitant sedation.

As further disclosed herein in Example V, several α-2 agonists were assayed for α-2A/α-1A functional selectivity in in vitro cell-based assays. As shown in Table 4, Compound 2 was a highly α-2/α-1 selective agent and was more selective than brimonidine, as indicated by its higher α-1A/α-2A potency ratio. Furthermore, another α-2 agonist, Compound 1, was also highly α-2/α-1 selective, as evidenced by the undetectable level of α-1A activity observed for this compound in a cell-based functional assay. These results indicate that Compounds 1 and 2 are highly selective for activation of the α-2A receptor as compared to the α-1A receptor. Furthermore, although dexmeditomidine was more potent than brimonidine at the α-2A receptor, dexmeditomidine was less α-2A/α-1A selective than was brimonidine (see Tables 3 and 4).

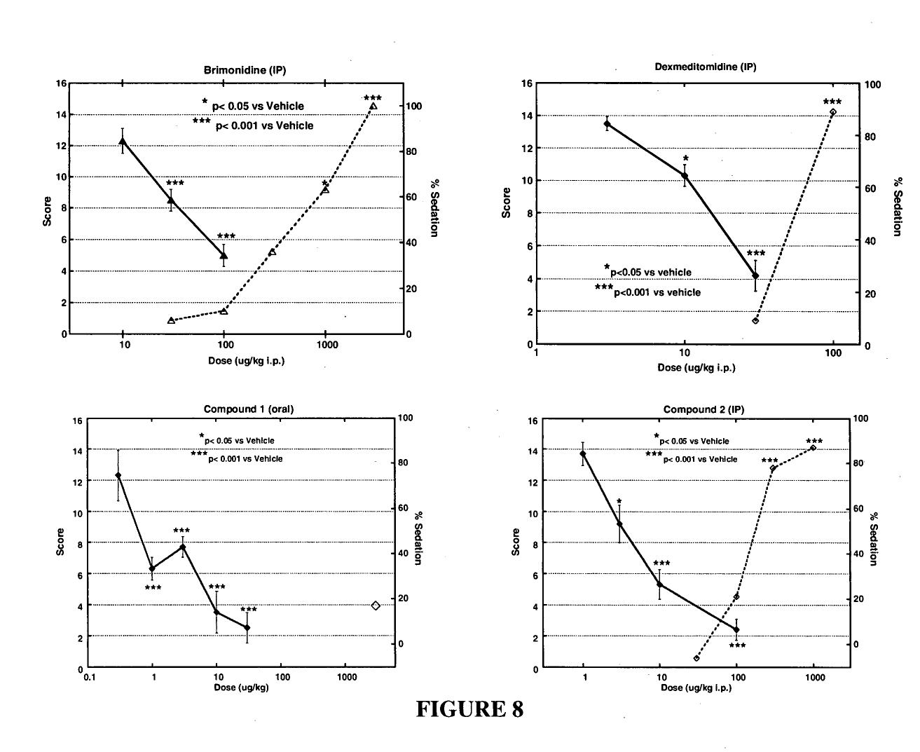

As additionally disclosed herein in Example V, the α-2/α-1 functional selectivity exhibited in cell-based assays inversely correlated with in vivo sedative activity at the therapeutic dose. As revealed in FIG. 8, α-2 agonists which were most selective for α-2/α-1 function in vitro were the same agonists which alleviated sulprostone-induced tactile sensitivity in the absence of concomitant sedation. In particular, Compound 1, administered orally at a dose of 1 μg/kg, produced a 50% reduction in sensitization (solid line, left axis), with less than 30% sedation (open diamond, right axis) at doses 100-fold and even 1000-fold greater than the 1 μg/kg effective dose (see FIG. 8, lower left panel). Similar results were obtained with intraperitoneal dosing. Furthermore, intraperitoneal administration of Compound 2 also produced more than a 50% reduction in sensitization at 10 μg/kg (solid line, left axis), with less than 30% sedation at a 10-fold greater dose. In sum, these results indicate that α-2A/α-1A adrenergic receptor selectivity of α-2 agonists in in vitro cell-based functional assays is inversely correlated with sedative activity in vivo at therapeutic doses following systemic or other peripheral administration. These results further indicate that particularly useful α-2 agonists are those exhibiting α-2A/α-1A adrenergic receptor functional selectivity similar to or better than that of brimonidine.

Based on these discoveries, the present invention provides methods of screening for an α-2A/α-1A selective agonist that prevents or alleviates sympathetically-enhanced conditions without concomitant sedation upon peripheral administration. Such screening methods are practiced by determining the functional selectivity of an agent for activating an α-2A receptor as compared to an α-1A receptor, where an agent which is highly selective for activating an α-2A receptor as compared to an α-1A receptor is an α-2A/α-1A selective agonist that prevents or alleviates sympathetically-enhanced conditions without concomitant sedation upon peripheral administration. As discussed further herein, such α-2A/α-1A selective agonists are also useful for preventing or alleviating neurological conditions, ocular conditions, chronic pain and other conditions without concomitant sedation.

In one embodiment, the invention provides a method of screening for an α-2A/α-1A selective agonist that prevents or alleviates sympathetically-enhanced conditions without concomitant sedation upon peripheral administration by (a) determining potency, activity or EC₅₀ of an agent at an α-2A receptor; and (b) determining potency, activity or EC₅₀ of the agent at an α-1A receptor, where an agent which has an α-1A efficacy less than that of brimonidine or a ratio of α-1A/α-2A potency greater than that of brimonidine is an α-2A/α-1A selective agonist that prevents or alleviates sympathetically-enhanced conditions without concomitant sedation. In a method of the invention, the selective agonist identified can have, without limitation, an α-1A efficacy less than that of brimonidine, or an α-1A/α-2A EC₅₀ ratio which is at least 30% greater than the α-1A/α-2A EC₅₀ ratio of brimonidine, two-fold greater than the α-1A/α-2A EC₅₀ ratio of brimonidine, five-fold greater than the α-1A/α-2A EC₅₀ ratio of brimonidine or ten-fold greater than the α-1A/α-2A EC₅₀ ratio of brimonidine. In further embodiments, a selective agonist useful in the invention has an α-1A/α-2A EC₅₀ ratio which is at least twenty-fold, thirty-fold, forty-fold, fifty-fold, sixty-fold, seventy-fold, eighty-fold, ninety-fold or 100-fold greater than the α-1A/α-2A EC₅₀ ratio of brimonidine.

Any of a variety of assays are useful in the screening methods of the invention. In one embodiment; potency, activity or EC₅₀ of an agent at an α-2A receptor is determined by assaying for inhibition of adenylate cyclase activity. As non-limiting examples, inhibition of adenylate cyclase activity can be assayed, for example, in PC12 cells stably expressing an α-2A receptor such as a human α-2A receptor. In another embodiment, potency, activity or EC₅₀ of the agent at an α-1A receptor is determined by assaying for intracellular calcium. As non-limiting examples, intracellular calcium can be assayed in HEK293 cells stably expressing a α-1A receptor such as a bovine α-1A receptor.

Agonist selectivity can be characterized using any of a variety of routine functional assays, for example, in vitro cell-based assays which measure the response of an agent proximal to receptor activation. Useful assays include, without limitation, in vitro assays such as cyclic AMP assays or GTPγS incorporation assays for analyzing function proximal to α-2 receptor activation (Shimizu et al., J. Neurochem. 16:1609-1619 (1969); Jasper et al., Biochem. Pharmacol. 55: 1035-1043 (1998); and intracellular calcium assays such as FLIPR assays and detection of calcium pulses by fluo-3 for analyzing function proximal to α-1 receptor activation (Sullivan et al., Methods Mol. Biol. 114:125-133 (1999); Kao et al., J. Biol. Chem. 264:8179-8184 (1989)). α-2A selectivity assays based on inhibition of forskolin-induced cAMP accumulation in PC12 cells stably expressing an α-2A receptor, and increases in intracellular calcium in HEK293 cells stably expressing an α-1A receptor, are disclosed herein in Example II below. Additional useful assays include, without limitation, inositol phosphate assays such as scintillation proximity assays (Brandish et al., Anal. Biochem. 313:311-318 (2003)); assays for β-arrestin GPCR sequestration such as bioluminescence resonance energy transfer assays (Bertrand et al., J. Receptor Signal Transduc. Res. 22:533-541 (2002)); and cytosensor microphysiometry assays (Neve et al., J. Biol. Chem. 267:25748-25753 (1992)). These and additional assays for proximal α-2 and α-1 receptor function are routine and well known in the art.

As a non-limiting example, a GTPγS assay is an assay useful for determining the functional selectivity of an agent for activating an α-2A receptor as compared to an α-1A receptor in the methods of the invention. α-2 adrenergic receptors mediate incorporation of guanosine 5′-O-(gamma-thio) triphosphate ([³⁵S]GTPγS) into G-proteins in isolated membranes via receptor-catalyzed exchange of [³⁵S]GTPYS for GDP. An assay based on [³⁵S]GTPγS incorporation can be performed essentially as described in Jasper et al., supra, 1998. Briefly, confluent cells treated with an agent to be tested are harvested from tissue culture plates in phosphate buffered saline before centrifuging at 300×g for five minutes at 4° C. The cell pellet is resuspended in cold lysis buffer (5 mM Tris/HCl, 5 mM EDTA, 5 mM EGTA, 0.1 mM PMSF, pH 7.5) using a Polytron Disrupter (setting #6, five seconds), and centrifuged at 34,000×g for 15 minutes at 4° C. before being resuspended in cold lysis buffer and centrifuged again as above. Following the second wash step, aliquots of the membrane preparation are placed in membrane buffer (50 mM Tris/HCl, 1 mM EDTA, 5 mM MgCl₂, and 0.1 mM PMSF, pH 7.4) and frozen at −70° C. until used in the binding assay.

GTPγS incorporation is assayed using [³⁵S]GTPγS at a specific activity of 1250 Ci/mmol. Frozen membrane aliquots are thawed and diluted in incubation buffer (50 mM Tris/HCl, 5 mM MgCl₂, 100 mM NaCl, 1 mM EDTA, 1 mM DTT, 1 mM propranolol, 2 mM GDP, pH 7.4) and incubated with radioligand at a final concentration of 0.3 nM at 25° C. for 60 minutes. After incubation, samples are filtered through glass fiber filters (Whatman GF/B, pretreated with 0.5% bovine serum albumin) in a 96-well cell harvester and rapidly washed four times with four mls of ice-cold wash buffer (50 mM Tris/HCl, 5 mM MgCl₂, 100 mM NaCl, pH 7.5). After being oven dried, the filters are transferred to scintillation vials containing five mls of Beckman's Ready Protein® scintillation cocktail for counting. The EC₅₀ and maximal effect (efficacy) of the agent to be tested are then determined for the α-2A receptor.

It is understood that useful assays generally are performed using cells that naturally express significant levels of only a single α-adrenergic receptor subtype or using transfected cells that express significant levels of only a single recombinant α-adrenergic receptor subtype. As a non-limiting example, the adrenergic receptor can be a human receptor or homolog thereof having a similar pharmacology. As disclosed herein, the screening methods of the invention are preferably practiced with receptor-proximal assays, i.e. those in which receptor response is unamplified or amplified only minimally or those in which a rapid signal is assayed. Thus, one skilled in the art will prefer to use assays other than Receptor Selection and Amplification Technology (RSAT) assays and similar assays in which partial and full agonism are not well differentiated.

The therapeutic methods disclosed herein rely on an “α-2A/α-1A selective agonist,” which, as used herein, is a term which means a compound (1) having greater than 25% efficacy relative to brimonidine at one or more α-2 adrenergic receptors including the α-2A adrenergic receptor and (2) further having an α-1A efficacy less than that of brimonidine or a ratio of α-1A/α-2A potency greater than that of brimonidine. Such a compound can be selective for the α-2A adrenergic receptor, or can be non-selective. Thus, the term α-2A/α-1A selective agonist encompasses, without limitation, pan-α-2 agonists; α-2A selective agonists; and agonists that are specific for the α-2A adrenergic receptor. In particular embodiments, a method of the invention utilizes an α-2A/α-1A selective agonist having greater than 30%, 40%, 50%, 60%, 70%, 80%, 90%, 100% or 200% efficacy relative to brimonidine at the α-2A receptor. In another embodiment, a method of the invention is practiced with an α-2A/α-1A selective agonist that lacks detectable efficacy at α-1A in a receptor-proximal assay such as the FLIPR assay for measuring intracellar calcium levels in HEK293 cells stably expressing a bovine α-1A receptor.

Efficacy, also known as intrinsic activity, is a measure of maximal receptor activation achieved by an agent. For the purposes of the screening methods of the invention, efficacy is preferably determined using any functional assay that does not significantly amplify receptor response. Efficacy can be represented as a ratio or percentage of the maximal effect of the agent to the maximal effect of a standard agonist for each receptor subtype. Brimonidine (UK14304) generally is used as the standard agonist for the α-2A, α-2B and α-2C receptors and is used as the standard herein where relative efficacy of an α-2 receptor is defined. Phenylephrine is an accepted standard agonist for the α-1A, α-1B and α-1D receptors and is used herein as the standard where relative efficacy of an α-1 receptor is defined.

As disclosed herein, an α-2A/α-1A selective agonist useful in the invention can be either selective or non-selective for the α-2A receptor as compared to other α-2 adrenergic receptors. Thus, an α-2A/α-1A selective agonist can be a pan-α-2 agonist or an agonist that is selective or specific for the α-2A receptor, provided that the agonist has an α-1A efficacy less than that of brimonidine or a ratio of α-1A/α-2A potency greater than that of brimonidine as described above. In particular embodiments, a method of the invention utilizes an α-2A/α-1A selective agonist having greater than 30%, 40%, 50%, 60%, 70%, 80%, 90%, 100% or 200% efficacy relative to brimonidine at the α-2A adrenergic receptor. Exemplary α-2A/α-1A selective agonists useful in the therapeutic methods of the invention include Compounds 1 and 2 disclosed herein. Pharmaceutically acceptable salts, esters, amides, sterioisomers and racemic mixtures of Compound 1 or Compound 2 also are useful in the invention. It is understood that, in addition to α-2A agonist activity, an α-2A/α-1A selective agonist useful in the invention may optionally have agonist or antagonist activity at one or more additional adrenergic or other receptors, provided that the selective agonist satisfies the criteria set forth above in regard to the α-1A receptor.

An α-2A/α-1A selective agonist useful in the invention can be a pan-α-2 agonist, which, as used herein, is term that means an agent having greater than 25% efficacy relative to brimonidine at each of the α-2A, α-2B and α-2C adrenergic receptors. In particular embodiments, a method of the invention is practiced with an α-2A/α-1A selective agonist which is a pan-α-2 agonist having greater than 30%, 40%, 50%, 60%, 70%, 80%, 90%, 100% or 200% efficacy relative to brimonidine at the α-2A, α-2B and α-2C adrenergic receptors. It is understood that the efficacy of a pan-α-2 agonist can be different at the various α-2 receptors; as a non-limiting example, a pan-α-2 agonist can have greater than 80% efficacy at the α-2A receptor, greater than 25% efficacy at the α-2B receptor and greater than 25% efficacy at the α-2C receptor.

In a screening method of the invention, the α-1A efficacy or ratio of α-1A/α-2A potencies of an agent, or both, is compared to that of brimonidine. As used herein, the term “brimonidine” means a compound having the formula 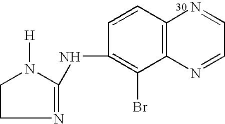
or a pharmaceutically acceptable derivative thereof. The term brimonidine encompasses, without limitation, 5-bromo-6-(2-imidazolin-2-ylamino) quinoxaline D-tartrate (1:1), Alphagan™ and UK14304. Brimonidine, and pharmaceutically acceptable derivatives thereof can be purchased from commercial sources or prepared by routine methods, for example, as described in U.S. Pat. No. 6,323,204.

Therapeutic methods based on α-2A/α-1A selective agonists also are provided herein. The present invention provides, for example, methods of preventing or alleviating a sympathetically-enhanced condition without concomitant sedation by peripherally administering to a subject an effective amount of an α-2A/α-1A selective agonist, thereby preventing or alleviating the sympathetically-enhanced condition without concomitant sedation, where the selective agonist has an α-1A efficacy less than that of brimonidine or a ratio of α-1A/α-2A potency greater than that of brimonidine. In a method of the invention, the selective agonist can have, without limitation, an α-1A efficacy less than that of brimonidine, or an α-1A/α-2A EC₅₀ ratio which is at least 30% greater than that of brimonidine, two-fold greater than that of brimonidine or ten-fold greater than that of brimonidine. Any of a variety of sympathetically-enhanced conditions can be prevented or alleviated without concomitant sedation according to a method of the invention including, without limitation, sensory hypersensitivity, for example, sensory hypersensitivity associated with fibromyalgia or headache such as migraine; gastrointestinal diseases such as irritable bowel syndrome and dyspepsia; dermatological conditions such as psoriasis; cardiovascular disorders; tachycardias; disorders of peripheral vasoconstriction such as Raynaud's Syndrome and scleroderma; panic attack; metabolic disorders such as type II diabetes, insulin-resistance and obesity; disorders of muscle contraction including disorders of skeletal muscle contraction, disorders of smooth muscle contraction, spasticity, and disorders of muscle contraction associated with tension-type headache; behavioral disorders; and sexual dysfunction. In one embodiment, the symapthetically-enhanced condition is a condition other than sympathetically maintained pain, which is any pain that can be relieved by sympathetic blockade. As non-limiting examples, the α-2A/α-1A selective agonist can be peripherally administered using oral administration, or using topical administration such as via a patch. In one embodiment, an effective amount of the α-2A/α-1A selective agonist is systemically administered to a subject to prevent or alleviate the sympathetically-enhanced condition without concomitant sedation.

In one embodiment, a method of the invention is useful for preventing or alleviating sensory hypersensitivity associated with headache without concomitant sedation. In a further embodiment, a method of the invention is useful for preventing or alleviating sensory hypersensitivity associated with migraine without concomitant sedation. Migraine is a headache which plagues more than 10% of the population and which may be associated with a vascular component. In one embodiment, the methods of the invention prevent or alleviate an ocular hypersensitivity associated with migraine, for example, photophobia, without concomitant sedation. It is understood that the methods of the invention are useful for preventing or alleviating sensory hypersensitivity associated with any of a variety of forms of migraine including, but not limited to, migraine without aura (“MO”), migraine with aura (“MA”), migrainous disorders, and, as non-limiting examples, abdominal migraine, acute confusional migraine, basilar (basilar artery) migraine, hemiplegic or familial hemiplegic migraine, fulgurating migraine, ocular (ophthalmic) migraine, ophthalmoplegic migraine or retinal migraine. In addition, the methods of the invention can be useful for preventing or alleviating sensory hypersensitivity associated with a migraine equivalent, in which there is a migraine aura without headache. Migraine auras are the abnormal visual, motor, psychic, paresthesic or other neurologic abnormalities that accompany a migraine. See Elrington, J. Neurol. Neurosurg. Psychiatry 72 Supple. II:ii10-ii15 (2002); Anderson, supra, 1994; Bennett and Plum, supra, 1996.

It is understood that the methods of the invention are useful for preventing or alleviating one or more of a variety of types of sensory hypersensitivity associated with migraine or other headache. Types of sensory hypersensitivity include, but are not limited to, nausea; vomiting; diarrhea; photophobia (light intolerance); and phonophobia (noise intolerance). Types of sensory hypersensitivity further include, without limitation, visual abnormalities such as bright flashing lights (scintillation or fortification scotomata) or a monocular (retinal) visual abnormality or hemianoptic loss of vision; paresthesia (abnormal touch sensation) such as unilateral paresthesia; aphasia (loss of speech or comprehension); hemiparesis (muscular weakness or incomplete paralysis on one side of the body); hemisensory defect; and vertigo, ataxia (loss of muscular coordination) or diplopia. It is understood that the methods of the invention can be useful for preventing or alleviating one of these or other types of sensory hypersensitivity occurring prior to, during, or subsequent to migraine or other headache, or occurring in the absence of headache, for example, as part of a migraine equivalent.

The methods of the invention also can be useful for preventing or alleviating any of a variety of types of sensory hypersensitivity associated with disorders other than headache, for example, fibromyalgia, which is also known as fibrositis. Fibromyalgia is a disorder involving chronic, widespread musculoskeletal pain and tenderness at multiple sites in the absence of signs of connective tissue or other musculoskeletal disease. In particular, fibromyalgia is defined by pain or tenderness at 11 of 18 or more sites specified by the American College of Rheumatology. Fibromyalgia frequently is associated with disturbed sleep, chronic fatigue, headaches and irritable bowel syndrome (Bennett and Plum, supra, 1996).

A variety of types of sensory hypersensitivity can be associated with fibromyalgia and can be prevented or alleviated without concomitant sedation according to a method of the invention, including, without limitation, hypersensitivity to light, noise, touch or odors, cold or heat intolerance, nausea or allergic-like symptoms such as rhinitis, itching, or rash in the absence of a true allergy. One skilled in the art understands that the methods of the invention can be useful for preventing or alleviating any of these or other types of sensory hypersensitivity associated with fibromyalgia without concomitant sedation.

A sympathetically-enhanced condition to be prevented or alleviated without concomitant sedation according to a method of the invention also can be a gastrointestinal disease. Inflammatory bowel disease (IBD) and irritable bowel syndrome (IBS) are gastrointestinal diseases which affect one-half of all Americans during their lifetime, at a cost of greater than $2.6 billion dollars for inflammatory bowel disease and greater than $8 billion dollars for irritable bowel syndrome. The frequency or severity of visceral hypersensitivity associated with these and other gastrointestinal diseases is exacerbated by stress. As disclosed herein, the methods of the invention can be useful for preventing or alleviating a gastrointestinal disease without concomitant sedation, including gastrointestinal diseases such as, without limitation, ulcerative colitis (UC), Crohn's disease (CD), irritable bowel syndrome and dyspepsia.

The gastrointestinal disease dyspepsia is often classified as a biopsychosocial disorder and is generally characterized, at least in part, by epigastric discomfort following meals. In addition to postprandial upper abdominal discomfort or pain, dyspepsia can be characterized by early satiety, nausea, vomiting, abdominal distension, bloating, or anorexia in the absence of organic disease (Thumshirn, Gut 51 Suppl. 1: i63-66 (2002); Anderson, Dorland's Illustrated Medical Dictionary 28^{th} Edition, W.B. Saunder's Company, Philadelphia (1994)). As used herein, the term “dyspepsia” means any form of impaired digestion. Any of a variety of types of dyspepsia can be prevented or alleviated without concomitant sedation according to a method of the invention including, without limitation, acid dyspepsia, which is associated with excessive acidity of the stomach; appendicular dyspepsia, also known as appendix dyspepsia, in which dyspeptic symptoms accompany chronic appendicitis; catarrhal dyspepsia, which is accompanied by gastric inflammation; chichiko dyspepsia, a condition of farinaceous malnutrition found in poorly nourished infants; cholelithic dyspepsia, which involves sudden dyspeptic attacks associated with gallbladder disturbance; colonic dyspepsia, which involves a functional disturbance of the large intestine; fermentive dyspepsia, which is characterized by fermentation of ingested food; flatulent dyspepsia, which is associated with the formation of gas in the stomach and often involves upper abdominal discomfort accompanied by frequent belching; gastric dyspepsia, which originates in the stomach; and intestinal dyspepsia, which originates in the intestines. It is understood that these and other mildly or acutely symptomatic forms of the condition are included in the definition of “dyspepsia” as used herein. In one embodiment, the methods of the invention are used to prevent or alleviate a dyspepsia other than one associated with gastric inflammation.

The invention further provides a method of preventing or alleviating a dermatological condition without concomitant sedation. Any of a variety of inflammatory and non-inflammatory dermatological conditions can be prevented or alleviated by a method of the invention such as, without limitation, inflammatory dermatological conditions including any of a variety of forms of acute or chronic dermatitis such as psoriasis, allergic dermatitis such as allergic contact dermatitis, atopic dermatitis, dermatitis calorica, contact dermatitis, cosmetic dermatitis, eczema, exfoliative dermatitis, factitial dermatitis, irritant dermatitis, lichen simplex chronicus, marine dermatitis, neurodermatitis, perioral dermatitis, phototoxic dermatitis, seborrheic dermatitis, stasis dermatitis and dermatitis vegetans. Dermatological conditions to be prevented or alleviated without concomitant sedation according to a method of the invention further include non-inflammatory dermatological conditions, which are any dermatosis or other skin disease or condition that is not caused or accompanied by inflammation. As non-limiting examples, non-inflammatory dermatological condition which can be prevented or alleviated without concomitant sedation according to a method of the invention encompass non-inflammatory dermatoses including non-inflammatory blistering diseases such as epidermolysis bullosa and porphyria; ichthyosis; keratosis pilaris; juvenile plantar dermatosis (JPD); lichen plantus dermatosis; and xerosis. One skilled in the art understands that these and other inflammatory and non-inflammatory dermatological conditions known in the art can be prevented or alleviated without concomitant sedation according to a method of the invention.

In one embodiment, a method of the invention prevents or alleviates psoriasis without concomitant sedation. Psoriasis is a common chronic, squamous dermatosis with a fluctuating course. Principle histological findings include Munro abscesses and spongiform pustules. Rounded, circumscribed, erythematous, dry, scaling patches, covered by grayish white or silvery white, umbilicated, and lamellar scales such as on extensor surfaces, nails, scalp, genitalia, and the lumbrosacral region may also be present. Forms of psoriasis which can be prevented or alleviated without concomitant sedation by a method of the invention include, yet are not limited to, annular psoriasis; arthritic psoriasis; Barber's psoriasis; psoriasis buccalis, circinate psoriasis, discoid psoriasis, erthrodermic psoriasis, exfoliative psoriasis, psoriasis figurata, flexural psoriasis (inverse psoriasis, sebborrheic psoriasis, volar psoriasis), follicular psoriasis, guttate psoriasis, psoriasis inveterata, psoriasis linguae, ostraceous psoriasis (psoriasis rupioides), palmar psoriasis, and localized and generalized pustular psoriasis.

A sympathetically-enhanced condition to be prevented or alleviated according to a method of the invention also can be a disorder of peripheral vasoconstriction such as, without limitation, Raynaud's Syndrome, also known as Raynaud's phenomenon or Raynaud's disease. Raynaud's Syndrome is characterized by intermittent bitlaterial attacks of ischemia of the fingers or toes, and sometimes of the ears or nose, generally accompanied by severe pallor and often by paresthesia or pain. Raynaud's Syndrome and other diseases of peripheral vasoconstriction can be sympathetically-enhanced, for example, triggered by emotional stimuli or cold. Mild Raynaud's is common and is not usually a harbinger of clinically important disability. However, more serious forms of Raynaud's can be the primary cause of poor health or can be associated with an underlying disorder such as a systemic rheumatic condition or systemic sclerosis (Block and Sequeira, Lancet 357:2042-2048 (2001); Wigley, N. Eng. J. Med. 347:1001-1018 (2002); and Pope, Cochrane Database Syst. Rev. CD000956 (2000)). Mild and severe forms of Raynaud's Syndrome, as well as other disorders of peripheral vasoconstriction including, without limitation, scleroderma (WO 00/76502) also can be prevented or alleviated using an α-2/α-1 selective agonist as disclosed herein.

The methods of the invention also can be useful for preventing or alleviating tachycardia without concomitant sedation. As used herein, the term “tachycardia” means excessive rapidity of heart rate and includes tachyarrhymthias. In adults, the term tachycardia generally refers to a heart rate of greater than 100 beats per minute. The term tachycardia encompasses tachycardias secondary to a variety of disorders including, without limitation, paroxysmal tachycardia, in which the tachycardia is of sudden onset and cessation and is either ventricular or supraventricular, and nonparoxysmal tachycardia, which is a tachycardia of slow onset, generally with a heart rate of 70 to 130 beats per minute. In one embodiment, the invention provides a method of preventing or alleviating, without concomitant sedation, a tachycardia other than a tachycardia associated with myocardial ischemia. In another embodiment, the invention provides a method of preventing or alleviating, without concomitant sedation, an automatic tachycardia other than one associated with myocardial ischemia. In still further embodiments, a method of the invention prevents or alleviates, without concomitant sedation, tachycardia in an adult subject, or tachycardia in a child.

Tachycardias to be prevented or alleviated without concomitant sedation according to a method of the invention include those originating from any part of the heart such as ventricular tachycardias and supraventricular tachycardias, which can be classified, for example, into atrial and junctional (nodal) tachycardias. Thus, the methods of the invention can be useful for preventing or alleviating, without limitation, ventricular tachycardias, which are abnormally rapid ventricular rhythms with aberrant ventricular excitation, often in excess of 150 beats per minutes, generated within the ventricle and sometimes occurring in conjunction with atrioventricular dissociation. The methods of the invention further can be useful for preventing or alleviating supraventricular tachycardias (SVT), which are regular tachycardias in which the point of stimulation is located above the bundle branches such as in the sinus node, atria or atrioventricular junction or which arise from a large reentrant circuit including both atrial and ventricular sites. In one embodiment, a method of the invention is used to prevent or alleviate an atrial tachycardia, which is characterized by a rapid cardiac rate generally between 160 and 190 beats per minutes and which originates from an atrial locus; such tachycardias include, but are not limited to, paroxysmal atrial tachycardias. In another embodiment, a method of the invention is used to prevent or alleviate a junctional tachycardia, which is a tachycardia arising in response to impulses originating in the atrioventricular junction and which is generally characterized by a heart rate greater than 75 beats per minute. Junctional tachycardias include nonparoxysmal and paroxysmal junctional tachycardias, such as junctional tachycardias resulting from reentry or enhanced automaticity. It is understood that the methods of the invention also can be used to prevent or alleviate a variety of other tachycardias including, without limitation, double tachycardias, in which two types of ectopic tachycardia are involved; sinus tachycardias, which originate in the sinus node and can be associated with shock, hypotension, congestive heart failure or fever; orthostatic tachycardia, which is characterized by a disproportionate rapidity of heart rate upon rising from a reclining to a standing position; and chaotic atrial tachycardia, which is characterized by atrial rates of 100 to 130 beats per minute, markedly variable P wave morphology and irregular P-P intervals (Anderson, supra, 1994).

One skilled in the art understands that tachycardias to be prevented or alleviated without concomitant sedation according to a method of the invention can be associated with a disorder such as pulmonary disease, diabetes, or surgical trauma and can occur, for example, in the elderly. As non-limiting examples, chaotic atrial tachycardia (multifocal atrial tachycardia) can be present in patients with chronic obstructive pulmonary disease, patients with diabetes, and in the elderly. As a further non-limiting example, nonparoxysmal junctional tachycardia can be associated with surgical trauma. One skilled in the art understands that these and other automatic and other tachycardias can be prevented or alleviated without concomitant sedation according to a method of the invention.

The methods of the invention also can be useful for preventing or alleviating a panic attack without concomitant sedation. Panic attacks are common disorders with a prevalence of around 3% in the general population (Potokar and Nutt, Int. J. Clin. Pract. 54: 110-114 (2000)). Panic disorder involving recurrent panic attacks is typically observed in young adults, with an average age of onset of 24 years, and is more common in females than in males. The term “panic attack,” as used herein, means a discrete period of intense fear or discomfort accompanied by one or more of the following symptoms: accelerated heart rate or palpitation; chest pain; chills or hot flushes; derealization or depersonalization; fear of dying; fear of losing control or going crazy; dizziness or faintness; feelings of choking; nausea or abdominal distress; paraesthesia; sensations of shortness of breath or smothering; sweating; or trembling or shaking. A panic attack typically begins with a sudden onset of intense apprehension or fear and generally has a duration of about 5 to 20 minutes. The term panic attack encompasses both full-blown and limited-symptom attacks; full-blown attacks involve four or more of the above symptoms while limited-symptom attacks involve fewer than four symptoms. A method of the invention can prevent or alleviate the severity of one or any combination of the attendant symptoms described above without concomitant sedation. In one embodiment, a method of the invention entirely prevents the panic attack.

Some patients with panic attacks develop “panic disorder,” which also can be prevented or alleviated without concomitant sedation according to a method of the invention. The term panic attack, as used herein, encompasses panic disorder, which is defined as recurrent panic attacks in conjunction with persistent concern over additional episodes or the consequences of the attacks or a notable change in behavior experienced for at least one month following one or more panic attacks. In some cases, panic disorder is associated with other psychiatric conditions such as depression.

The central sympathetic nervous system can play a critical role in the development of metabolic disorders such as the insulin-resistance and hypertension which characterize type II diabetes (Rocchini et al., Hypertension 33[part II]:548-553 (1999)). Thus, further provided herein is a method of preventing or alleviating a metabolic disorder without concomitant sedation. The metabolic disorder can be, without limitation, type II diabetes, insulin-resistance, obesity, or a disorder characterized by hypertension, hyperlipidemia and insulin-resistance.

The methods of the invention also can be useful for preventing or alleviating any of a variety of disorders of muscle contraction without concomitant sedation. Disorders of muscle contraction are conditions that result, at least in part, from inappropriate muscle contraction and include, without limitation, disorders of skeletal muscle contraction, disorders of smooth muscle contraction, disorders of muscle contraction associated with a gland, and disorders of cardiac muscle contraction such as congestive heart failure; these and other disorders of muscle contraction to be prevented or alleviated without concomitant sedation according to a method of the invention include those in which the myocytes are innervated as well as those in which the myocytes are not innervated. As non-limiting examples, a method of the invention can be useful for preventing or alleviating a disorder of muscle contraction such as back or other muscle spasm; muscle contraction associated with cystitis; muscle contraction associated with non-bacterial prostatitis; muscle contraction associated with teeth grinding; muscle contraction associated with tension type headache; and muscle contraction associated with congestive heart failure.

In one embodiment, the disorder of muscle contraction is muscle spasm. As used herein, the term “spasm” means a sudden, involuntary contraction of a muscle or a group of muscles, accompanied by pain and interference with function. A spasm can produce, for example, involuntary movement or distortion. In one embodiment, a method of the invention prevents or alleviates a back spasm without concomitant sedation.

Muscle contraction associated with cystitis also is a disorder of muscle contraction which can be prevented or alleviated without concomitant sedation according to a method of the invention. As used herein, the term “cystitis” means inflammation of the urinary bladder. The term cystitis encompasses, yet is not limited to, allergic cystitis, bacterial cystitis, acute catarrhal cystitis, cystic cystitis, diphtheritic (croupous) cystitis, eosinophilic cystitis, exfoliative cystitis, cystitis follicularis, cystitis glandularis, incrusted cystitis, chronic interstitial (panmural, submucous) cystitis, mechanical cystitis, cystitis papillomatosa and cystitis senilis feminarum. See, for example, Anderson, supra, 1994. Cystitis can be accompanied by one or more of the following clinical symptoms: frequent urination, burning on urination, suprapubic discomfort, lassitude, cloudy or blood-tinged urine and sometimes low-grade fever (Bennett and Plum (Eds.), Cecil Textbook of Medicine Sixth Edition, W.B. Saunders Company, Philadelphia 1996). One skilled in the art understands that the muscle contraction associated with any of these or other forms of mild, severe, acute or chronic cystitis can be prevented or alleviated without concomitant sedation according to a method of the invention.

Muscle contraction associated with non-bacterial prostatitis also is a disorder of muscle contraction which can be prevented or alleviated without concomitant sedation according to a method of the invention. Symptoms of prostatic inflammation are experienced by about 50% of men in adult life; of these, about 95% result from factors other than bacterial infection. As used herein, the term “non-bacterial prostatitis” is synonymous with “abacterial prostatitis” and means inflammation of the prostate not resulting from bacterial infection. Non-bacterial prostatitis encompasses, yet is not limited to, chronic non-bacterial prostatitis, allergic or eosinophilic prostatitis and non-specific granulomatous prostatitis. It is understood that the term non-bacterial prostatitis includes, without limitation, prostatitis of unknown etiology characterized by abnormal expressed prostatic secretions (EPS) and normal bacterial cultures. It is understood that muscle contraction associated with these and other forms of mild, severe, acute or chronic non-bacterial prostatitis can be prevented or alleviated without concomitant sedation according to a method of the invention.

Muscle contraction associated with tension type headache (TTH) also can be prevented or alleviated without concomitant sedation according to a method of the invention. Tension type headaches are a common form of headache affecting as many as 90% of adult Americans. As used herein, the term “tension type headache” means a headache caused, at least in part, by muscle contraction, which may be triggered, for example, by stress or exertion. The term “tension type headache” encompasses episodic and chronic headache and includes, but is not limited to, common tension headache. Tension type headache generally involves the posterior of the head and neck, although it may also appear at the top or front of the skull. Tension type headache further is generally generally characterized by symmetry and a non-disabling severity. Although not all may be present, diagnostic features of tension type headache include bilateral pain; mild to moderate severity; pressing-like character with a stable profile; accentuation as the day progresses; possible high frequency such as daily or continuously; and relative rarity of migrainous features such as nausea, photosensitivity, phonosensitivity and aggravation by physical activity such as head movement.

Tension type headache results from tightening of muscles of the face, neck and scalp due, for example, to stress, overwork, eyestrain or poor posture. Such a headache can last for days or weeks and can cause pain of varying intensity. Tension type headache occurring over an extended period of time such as several weeks or months is denoted chronic tension headache and is encompassed by the term tension type headache as used herein.

Tension type headache can be distinguished from migraine by the absence of vascular features and symptoms such as nausea, vomiting, sensitivity to light and the absence of an aura (Spira, Austr. Family Phys. 27: 597-599 (1998). The term tension type headache, which refers to headache without a significant vascular component, is used herein in contradistinction to tension-vascular headache, cluster headache, migrainous headache and other headaches with a major vascular component. However, the methods of the invention also can be useful for preventing or alleviating sensory hypersensitivity associated with other headaches including, but not limited to, cervicogenic headache, post-traumatic headache, cluster headache and temporomandibular joint disorder (TMJ).

The methods of the invention further can be useful for preventing or alleviating a behavioral disorder without concomitant sedation. In one embodiment, the disorder is a stress-associated behavioral disorder, which is any behavioral disorder which is induced or exacerbated by stress. As non-limiting examples, a stress-associated behavioral disorder can be a compulsive or repetitive detrimental behavior which is induced or exacerbated by stress such as over-eating or obesity, obsessive compulsive disorder (OCD), tics, Tourette syndrome (TS), alcohol use, drug use, gambling, self-inflicted injurious behavior such as scratching or hair-pulling, or sexual impotency or arousal. In one embodiment, the stress-associated behavioral disorder is a disorder other than drug use. In another embodiment, the stress-associated behavioral disorder is a disorder other than drug or alcohol use.

The methods of the invention further can be useful for preventing or alleviating a psychiatric disorder without concomitant sedation. In one embodiment, the psychiatric disorder is one which is induced or exacerbated by stress. As a non-limiting example, the methods of the invention can be used to prevent or alleviate schizophrenia without concomitant sedation.

The methods of the invention are useful for preventing or alleviating a variety of sympathetically-enhanced conditions, neurological conditions, ocular conditions, types of chronic pain and other conditions disclosed herein without concomitant sedation. The term “alleviating,” as used herein, means reducing by at least about 50% at least one symptom of the particular condition or type of chronic pain being treated.

Sedation is a term that means a reduction in motor activity. The phrase “without concomitant sedation,” as used herein in reference to an agonist, means that, upon peripheral administration, the agonist produces less than about 30%-sedation at a dose 10-fold greater than the dose of agonist required to produce a 50% reduction of one or more symptoms of the particular condition or type of chronic pain being treated. For example, as shown in FIG. 8 (lower left panel), Compound 1 was administered orally at a dose of 1 μg/kg to produce a 50% reduction in sensitization score (solid line, left axis) with less than 30% sedation (open diamond, right axis) at doses 100-fold and even 1000-fold greater than the 1 μg/kg effective dose. Furthermore, as shown in FIG. 8E, intraperitoneal administration of Compound 2 produced a more than 50% reduction in sensitization score at 10 μg/kg (solid line, left axis), with less than 30% sedation at a dose 10-fold higher (100 μg/kg) than this effective dose. Thus, Compounds 1 and 2 have effective therapeutic activity “without concomitant sedation.” In contrast, dexmeditomidine was completely sedating at a dose 10-fold greater than the dose required to produce a 50% reduction in sensitization score.

As non-limiting examples, the dose required to produce about 30% sedation (reduction in motor activity) can be at least 25-fold greater than, 50-fold greater than, 100-fold greater than, 250-fold greater than, 500-fold greater than, 1000-fold greater than, 2500-fold greater than, 5000-fold greater than, or 10,000-fold greater than the dose required to produce a 50% reduction in one or more symptoms of the particular condition or type of chronic pain being treated. Methods for determining the extent of a reduction in a symptom as well as the extent of sedation are described herein and further are well known in the art.

Further provided herein is a method of preventing or alleviating chronic pain without concomitant sedation by peripherally administering to a subject an effective amount of an α-2A/α-1A selective agonist, thereby preventing or alleviating the chronic pain without concomitant sedation, where the selective agonist has an α-1A efficacy less than that of brimonidine or a ratio of α-1A/α-2A potency greater than that of brimonidine. In a method of the invention for preventing or alleviating chronic pain without concomitant sedation, the selective agonist can have, without limitation, an α-1A efficacy less than that of brimonidine, or an α-1A/α-2A EC₅₀ ratio which is at least 30% greater than that of brimonidine, two-fold greater than that of brimonidine or ten-fold greater than that of brimonidine.

Any of a variety of types of chronic pain can be prevented or alleviated without concomitant sedation according to a method of the invention, including, but not limited to, neuropathic pain such as neuropathic pain associated with diabetic neuropathy or post-herpetic neuralgia; chronic pain associated with cancer; post-operative pain; allodynic pain such as fibromyalgic pain; chronic pain associated with Complex Regional Pain Syndrome (CRPS); chronic visceral pain such as that associated with irritable bowel syndrome or dysmennorhea; chronic headache pain such as migraine pain, non-vascular headache pain, cluster headache pain or daily tension headache pain; and chronic muscle pain such as that associated with back spasm. The methods of the invention for preventing or alleviating chronic pain without concomitant sedation can be practiced using any of a variety of routes of peripheral administration including, but not limited to, oral administration and topical administration, for example, via a patch. In one embodiment, an effective amount of the α-2A/α-1A selective agonist is systemically administered to a subject to prevent or alleviate the chronic pain without concomitant sedation.

The term “chronic pain,” as used herein, means pain other than acute pain and includes, without limitation, neuropathic pain, visceral pain, inflammatory pain, headache pain, muscle pain and referred pain. It is understood that chronic pain is of relatively long duration, for example, several years and can be continuous or intermittent. Chronic pain is distinguished from acute pain, which is immediate, generally high threshold, pain brought about by injury such as a cut, crush, burn, or by chemical stimulation such as that experienced upon exposure to capsaicin, the active ingredient in chili peppers.

In one embodiment, the methods of the invention for preventing or alleviating chronic pain without concomitant sedation are used to treat “neuropathic pain,” which, as used herein, is a term that means pain resulting from injury to a nerve. Neuropathic pain is distinguished from nociceptive pain, which is the pain caused by acute tissue injury involving small cutaneous nerves or small nerves in muscle or connective tissue. In contrast to neuropathic pain, pain involving a nociceptive mechanism usually is limited in duration to the period of tissue repair and generally is relieved by available analgesic agents or opioids (Myers, Regional Anesthesia 20:173-184 (1995)). Chronic neuropathic pain can develop days or months following an initial acute tissue injury and can involve persistent, spontaneous pain as well as allodynia, which is a painful response to a stimulus that normally is not painful, or hyperalgesia, an accentuated response to a painful stimulus that usually is trivial such as a pin prick.

Any of a variety of types of neuropathic pain can be prevented or alleviated without concomitant sedation according to a method of the invention. As non-limiting examples, neuropathic pain can result from, or be associated with, a trauma, injury or disease of peripheral nerve, dorsal root ganglia, spinal cord, brainstem, thalamus or cortex. Neuropathic pain which can be prevented or alleviated without concomitant sedation by a method of the invention includes, without limitation, neuralgia such as post-herpetic neuralgia and trigeminal neuralgia; deafferentation pain; diabetic neuropathy; ischemic neuropathy; and drug-induced pain such as that accompanying taxol treatment. It is understood that the methods of the invention are useful in preventing or alleviating neuropathic pain without concomitant sedation regardless of the etiology of the pain and can, without limitation, be useful for preventing or alleviating neuropathic pain resulting from a peripheral nerve disorder such as neuroma; nerve compression; nerve crush or stretch or incomplete nerve transsection; or from a mononeuropathy or polyneuropathy. As still further non-limiting examples, the methods of the invention are useful for preventing or alleviating neuropathic pain which results from a disorder such as dorsal root ganglion compression; inflammation of the spinal cord; contusion, tumor or hemisection of the spinal cord; and tumors or trauma of the brainstem, thalamus or cortex.

As indicated above, the methods of the invention can be useful for preventing or alleviating neuropathic pain resulting from a mononeuropathy or polyneuropathy without concomitant sedation. A neuropathy is a functional disturbance or pathological change in the peripheral nervous system and is characterized clinically by sensory or motor neuron abnormalities. Mononeuropathies are neuropathies in which a single peripheral nerve is affected, while polyneuropathies are neuropathies in which several peripheral nerves are affected. The etiology of a neuropathy to be prevented or alleviated without concomitant sedation according to a method of the invention can be known or unknown. Known etiologies include, yet are not limited to, complications of a disease or toxic state such as diabetes, which is the most common metabolic disorder causing neuropathy, or irradiation, ischemia or vasculitis. Polyneuropathies that can be prevented or alleviated without concomitant sedation according to a method of the invention include, without limitation, those resulting from post-polio syndrome, diabetes, alcohol, amyloid, toxins, HIV, hypothyroidism, uremia, vitamin deficiencies, chemotherapy, ddC or Fabry's disease. It is understood that the methods of the invention can be used to prevent or alleviate these and other types of chronic neuropathic pain of known or unknown etiology.

As additional non-limiting examples, the methods of the invention can be useful for preventing or alleviating chronic pain associated with cancer, which is chronic pain caused by cancer or attendant to the treatment of cancer, for example, attendant to chemotherapy or radiation therapy; post-operative pain; allodynic pain such as fibromyalgic pain; chronic pain associated with Complex Regional pain Syndrome (CRPS); chronic visceral pain such as that associated with irritable bowel syndrome or dysmennorhea; chronic inflammatory pain resulting, for example, from spondylitis or arthritis such as rheumatoid arthritis, gouty arthritis, or osteoarthritis; chronic inflammatory pain resulting from chronic gastrointestinal inflammations such as Crohn's disease, ulcerative colitis, gastritis or irritable bowel disease; or other types of chronic inflammatory pain such as corneal pain or pain resulting from an autoimmune disease such as lupus erythematosus. The methods of the invention further can be used, without limitation, to treat chronic headache pain such as pain associated with migraines, non-vascular headaches, cluster headaches, tension headaches or chronic daily headaches; muscle pain including, but not limited to, that associated with back or other spasm; and the pain associated with substance abuse or withdrawal as well as other types of chronic pain of known or unknown etiology.

The present invention further provides a method of preventing or alleviating a neurological condition without concomitant sedation by peripherally administering to a subject an effective amount of an α-2A/α-1A selective agonist, thereby preventing or alleviating the neurological condition without concomitant sedation, where the selective agonist has an α-1A efficacy less than that of brimonidine or a ratio of α-1A/α-2A potency greater than that of brimonidine. In a method of the invention for preventing or alleviating a neurological condition without concomitant sedation, the selective agonist can have, without limitation, an α-1A efficacy less than that of brimonidine, or an α-1A/α-2A EC₅₀ ratio which is at least 30% greater than that of brimonidine, two-fold greater than that of brimonidine or ten-fold greater than that of brimonidine. Any of a variety of neurological conditions can be prevented or alleviated without concomitant sedation according to a method of the invention including both acute and chronic neurological conditions. As non-limiting examples, acute neurological conditions which can be prevented or alleviated without concomitant sedation include stroke; head or spinal cord trauma; and seizure. Chronic neurological conditions which can be prevented or alleviated without concomitant sedation according to a method of the invention include, but are not limited to, neurodegenerative diseases such as Alzheimer's disease; Parkinson's disease; Huntington's disease;
amyotrophic lateral sclerosis and multiple sclerosis; HIV-associated dementia and neuropathy; ocular diseases such as glaucoma, diabetic neuropathy and age-related macular degeneration; and schizophrenia, drug addiction, drug withdrawal, drug dependency, depression and anxiety. In the methods of the invention, acute and chronic neurological conditions can be prevented or alleviated by any of a variety of routes of peripheral administration including, yet not limited to, oral administration and topical administration, for example, via a patch. In one embodiment, an effective amount of the α-2A/α-1A selective agonist is systemically administered to a subject to prevent or alleviate the neurological condition without concomitant sedation.

The term “neurological condition” as used herein, encompasses all acute and chronic disorders which affect, at least in part, neurons. Thus, the term neurological condition encompasses, without limitation, hypoxia-ischemia (stroke); head and spinal cord injury; epilepsy; neurodegenerative disorders such as Parkinson's disease, Huntington's disease, Alzheimer's disease, amyotrophic lateral sclerosis or multiple sclerosis; optic neuropathies such as glaucoma, light-induced retinal degeneration such as photoreceptor degeneration, and macular degeneration; disorders of photoreceptor degeneration such as retinitis pigmentosa; metabolic, mitochondrial and infectious brain abnormalities such as encephalitis; and neuropathic pain (Lipton and Rosenberg, New Engl. J. Med. 330: 613 (1994)). Chronic neurological conditions include, yet are not limited to, neurodegenerative diseases such as Alzheimer's disease; Parkinson's disease; Parkinsonism; Huntington's disease; amyotrophic lateral sclerosis and multiple sclerosis; disorders of photoreceptor degeneration such as retinitis pigmentosa and light-induced retinal degeneration; macular degeneration of the retina and other ocular disorders such as glaucoma and diabetic retinopathy; HIV-associated dementia (acquired immunodeficiency syndrome dementia complex) and HIV-associated neuropathy; neuropathic pain syndromes such as causalgia or painful peripheral neuropathies; olivopontocerebellar atrophy; mitochondrial abnormalities and other biochemical disorders such as MELAS syndrome, MERRF, Leber's disease, Wernicke's encephalopathy, Rett syndrome, homocysteinuria, hyperhomocysteinemia, hyperprolinemia, nonketotic hyperglycinemia, hydroxybutyric aminoaciduria, sulfite oxidase deficiency, combined systems disease, lead encephalopathy; hepatic encephalopathy, Tourette's syndrome; drug addiction and drug dependency; drug withdrawal such as withdrawal from alcohol or opiates; and depression or anxiety syndromes.

An acute neurological condition is any neurological condition having a short and relatively severe course. As non-limiting examples, an acute neurologic condition which can be prevented or alleviated without concomitant sedation according to a method of the invention can be cerebral ischemia associated with stroke; hypoxia; anoxia; poisoning by carbon monoxide, manganese or cyanide; hypoglycemia; perinatal asphyxia; near death drowning; mechanical trauma to the nervous system such as trauma to the head or spinal cord; epileptic or other seizure; cardiac arrest; or cerebral asphyxia associated, for example, with coronary bipass surgery. Acute neurological conditions generally are distinguished from chronic neurological conditions, in which the neurological condition is of a relatively long duration, for example, several months or years.

Further provided herein is a method of preventing or alleviating an ocular condition without concomitant sedation by peripherally administering to a subject an effective amount of an α-2A/α-1A selective agonist, thereby preventing or alleviating the ocular condition without concomitant sedation, where the selective agonist has an α-1A efficacy less than that of brimonidine or a ratio of α-1A/α-2A potency greater than that of brimonidine. In a method of the invention for preventing or alleviating an ocular condition without concomitant sedation, the selective agonist can have, without limitation, an α-1A efficacy less than that of brimonidine, or an α-1A/α-2A EC₅₀ ratio which is at least 30% greater than that of brimonidine, two-fold greater than that of brimonidine or ten-fold greater than that of brimonidine. Ocular conditions to be prevented or alleviated without concomitant sedation according to a method of the invention include, without limitation, glaucoma; macular degeneration; and retinopathies such as diabetic retinopathy. As non-limiting examples, routes of peripheral administration useful in the invention include eye drops, intraocular implants, oral administration and topical administration such as via a patch. In another embodiment, an effective amount of the α-2A/α-1A selective agonist is systemically administered to a subject to prevent or alleviate the ocular condition without concomitant sedation.

A variety of ocular conditions can be prevented or alleviated without concomitant sedation according to a method of the invention. Such conditions include, without limitation, diabetic retinopathy; macular edema such as that associated with diabetes; conditions of retinal degeneration such as glaucoma, macular degeneration such as age-related macular degeneration (ARMD) and retinitis pigmentosa; retinal dystrophies; inflammatory disorders of the retina; vascular occlusive conditions of the retina such as retinal vein occlusions or branch or central retinal artery occlusions; retinopathy of prematurity; retinopathy associated with blood disorders such as sickle cell anemia; elevated intraocular pressure; ocular itch; damage following retinal detachment; damage or insult due to vitrectomy, retinal or other surgery; and other retinal damage including therapeutic damage such as that resulting from laser treatment of the retina, for example, pan-retinal photocoagulation for diabetic retinopathy or photodynamic therapy of the retina, for example, for age-related macular degeneration. Ocular conditions that can be prevented or alleviated without concomitant sedation according to a method of the invention further include, without limitation, genetic and acquired optic neuropathies such as optic neuropathies characterized primarily by loss of central vision, for example, Leber's hereditary optic neuropathy (LHON), autosomal dominant optic atrophy (Kjer disease) and other optic neuropathies such as those involving mitochondrial defects, aberrant dynamin-related proteins or inappropriate apoptosis; and optic neuritis such as that associated with multiple sclerosis, retinal vein occlusions or photodynamic or laser therapy. See, for example, Carelli et al., Neurochem. Intl. 40:573-584 (2002); and Olichon et al., J. Biol. Chem. 278:7743-7746 (2003). It is understood that these and other ocular abnormalities, especially those of the neurosensory retina, can be prevented or alleviated without concomitant sedation according to a method of the invention.

In addition to preventing or alleviating sympathetically-enhanced conditions, neurological conditions, ocular conditions and chronic pain, an α-2A/α-1A selective agonist can be used to prevent or alleviate other disorders without concomitant sedation. Such a disorder can be, for example, attention deficit disorder (ADHD/ADD), which is a disorder primarily characterized by inattention, distractibility and impulsiveness starting before the age of seven. Symptoms can include, without limitation, fidgeting and squirming, difficulty in remaining seated, easy distractability, difficulty awaiting one's turn, difficulty in refraining from blurting out answers, inability to follow instructions, excessive talking, and other disruptive behavior (Anderson, supra, 1994). Furthermore, while originally recognized in children, ADHD/ADD continues into adulthood in many individuals (see, for example, Block, Pediatr. Clin. North Am. 45:1053-1083 (1998); and Pary et al., Ann. Clin. Psychiatry 14:105-111 (2002)). One skilled in the art understands that a method of the invention can be useful for preventing or alleviating ADHD/ADD in children and adults having mild as well as severe forms of the disorder. Additional disorders to be prevented or alleviated according to a method of the invention include, without limitation, nasal congestion; diarrhea; urinary disorders such as hyperactive micturition and overactive bladder; congestive heart failure; or a psychosis such as a manic disorder. An α-2A/α-1A selective agonist also can be useful to prevent or alleviate one or more symptoms associated with anesthesia such as nausea, vomiting, shivering or panic; or to enhance memory and cognitive processes, without concomitant sedation.

It is understood that pharmaceutical compositions containing an effective amount of an α-2A/α-1A selective agonist can be useful in the methods of the invention for preventing or alleviating a sympathetically-enhanced condition, neurological condition, ocular condition or chronic pain without concomitant sedation. Such a pharmaceutical composition includes an α-2A/α-1A selective agonist and optionally includes an excipient such as a pharmaceutically acceptable carrier or a diluent, which is any carrier or diluent that has substantially no long term or permanent detrimental effect when administered to a subject. An excipient generally is mixed with an active α-2A/α-1A selective agonist, or permitted to dilute or enclose the selective agonist. A carrier can be a solid, semi-solid, or liquid agent that acts as an excipient or vehicle for the active selective agonist. Examples of solid carriers include, without limitation, pharmaceutical grades of mannitol, lactose, starch, magnesium stearate, sodium saccharin, polyalkylene glycols, talcum, cellulose, glucose, sucrose and magnesium carbonate. Suppository formulations can include, for example, propylene glycol as a carrier. Examples of pharmaceutically acceptable carriers and diluents include, without limitation, water, such as distilled or deionized water; saline; aqueous dextrose, glycerol, ethanol and the like. It is understood that the active ingredients can be soluble or can be delivered as a suspension in the desired carrier or diluent.

A pharmaceutical composition also can optionally include one or more agents such as, without limitation, emulsifying agents, wetting agents, sweetening or flavoring agents, tonicity adjusters, preservatives, buffers or anti-oxidants. Tonicity adjustors useful in a pharmaceutical composition include, but are not limited to, salts such as sodium acetate, sodium chloride, potassium chloride, mannitol or glycerin and other pharmaceutically acceptable tonicity adjustors. Preservatives useful in pharmaceutical compositions include, without limitation, benzalkonium chloride, chlorobutanol, thimerosal, phenylmercuric acetate, and phenylmercuric nitrate. Various buffers and means for adjusting pH can be used to prepare a pharmaceutical composition, including, but not limited to, acetate buffers, citrate buffers, phosphate buffers and borate buffers. Similarly, anti-oxidants useful in pharmaceutical compositions are well known in the art and include, for example, sodium metabisulfite, sodium thiosulfate, acetylcysteine, butylated hydroxyanisole and butylated hydroxytoluene. It is understood that these and other substances known in the art of pharmacology can be included in a pharmaceutical composition useful in the methods of the invention. See, for example, Remington's Pharmaceutical Sciences Mack Publishing Company, Easton, Pa. 16^{th} Edition 1980. Furthermore, a pharmaceutical composition containing an α-2A/α-1A selective agonist can optionally be administered in conjunction with one or more other therapeutic substances, in the same or different pharmaceutical composition and by the same or different routes of administration.

An α-2A/α-1A selective agonist is peripherally administered to a subject in an effective amount. Such an effective amount generally is the minimum dose necessary to achieve the desired prevention or alleviation of one or more symptoms of the sympathetically-enhanced condition, neurological condition, ocular condition or chronic pain, for example, that amount roughly necessary to reduce to tolerable levels the discomfort caused by the sympathetically-enhanced condition, neurological condition, ocular condition or chronic pain. Such a dose generally is in the range of 0.1-1000 mg/day and can be, for example, in the range of 0.1-500 mg/day, 0.5-500 mg/day, 0.5-100 mg/day, 0.5-50 mg/day, 0.5-20 mg/day, 0.5-10 mg/day or 0.5-5 mg/day, with the actual amount to be administered determined by a physician taking into account the relevant circumstances including the severity and type of sympathetically-enhanced condition, neurological condition, ocular condition or chronic pain; the age and weight of the patient; the patient's general physical condition; and the pharmaceutical formulation and route of administration. As discussed further below, suppositories and extended release formulations also can be useful in the methods of the invention, including, without limitation, dermal patches, formulations for deposit on or under the skin and formulations for intramuscular injection.

Ophthalmic compositions can be useful in the methods of the invention for preventing or alleviating an ocular condition without concomitant sedation. An ophthalmic composition contains an ophthalmically acceptable carrier, which is any carrier that has substantially no long term or permanent detrimental effect on the eye to which it is administered. Examples of ophthalmically acceptable carriers include, without limitation, water, such as distilled or deionized water; saline; and other aqueous media. An ophthalmic composition useful in the invention can include, for example, a soluble α-2A/α-1A selective agonist, or an α-2A/α-1A selective agonist as a suspension in a suitable carrier.

Topical ophthalmic compositions useful for preventing or alleviating an ocular condition include, without limitation, ocular drops, ocular ointments, ocular gels and ocular creams. Such ophthalmic compositions are easy to apply and deliver the active agonist effectively. Components of a non-limiting, exemplary topical ophthalmic composition are shown below in Table 1.

TABLE 1

| Ingredient | Amount (% W/V) |
|---|---|
| α-2A/α-1A selective agonist | about 0.0001 to |
| | about 0.1 |
| Preservative | 0-0.10 |
| Vehicle | 0-40 |
| Tonicity Adjustor | 1-10 |
| Buffer | 0.01-10 |
| pH Adjustor | q.s. pH 4.5-7.5 |
| antioxidant | As needed |
| Purified Water | As needed to make 100% |

A preservative can be included, if desired, in an ophthalmic composition useful in a method of the invention. Such a preservative can be, without limitation, benzalkonium chloride, chlorobutanol, thimerosal, phenylmercuric acetate, or phenylmercuric nitrate. Vehicles useful in a topical ophthalmic composition include, yet are not limited to, polyvinyl alcohol, povidone, hydroxypropyl methyl cellulose, poloxamers, carboxymethyl cellulose, hydroxyethyl cellulose and purified water.

A tonicity adjustor also can be included, if desired, in an ophthalmic composition administered to prevent or alleviate an ocular condition without concomitant sedation according to a method of the invention. Such a tonicity adjustor can be, without limitation, a salt such as sodium chloride, potassium chloride, mannitol or glycerin, or another pharmaceutically or ophthalmically acceptable tonicity adjustor.

Various buffers and means for adjusting pH can be used to prepare an ophthalmic composition useful in the invention, provided that the resulting preparation is ophthalmically acceptable. Such buffers include, but are not limited to, acetate buffers, citrate buffers, phosphate buffers and borate buffers. It is understood that acids or bases can be used to adjust the pH of the composition as needed. Ophthalmically acceptable antioxidants useful in preparing an ophthalmic composition include, yet are not limited to, sodium metabisulfite, sodium thiosulfate, acetylcysteine, butylated hydroxyanisole and butylated hydroxytoluene.

A method of the invention is practiced by peripherally administering to a subject an effective amount of an α-2A/α-1A selective agonist. As used herein in reference to an α-2A/α-1A selective agonist, the term “peripherally administering” or “peripheral administration” means introducing the α-2A/α-1A selective agonist into a subject outside of the central nervous system. Thus, peripheral administration encompasses any route of administration other than direct administration to the spine or brain.

An effective amount of an α-2A/α-1A selective agonist can be peripherally administered to a subject by any of a variety of means depending, for example, on the type of condition or chronic pain to be prevented or alleviated, the pharmaceutical formulation, and the history, risk factors and symptoms of the subject. Routes of peripheral administration suitable for the methods of the invention include both systemic and local administration. As non-limiting examples, an effective amount of an α-2A/α-1A selective agonist can be administered orally; parenterally; by subcutaneous pump; by dermal patch; by intravenous, intra-articular, subcutaneous or intramuscular injection; by topical drops, creams, gels or ointments; as an implanted or injected extended release formulation; or by subcutaneous minipump or other implanted device.

One skilled in the art understands that peripheral administration can be local or systemic. Local administration results in significantly more of an α-2A/α-1A selective agonist being delivered to and about the site of local administration than to regions distal to the site of administration. Systemic administration results in delivery of an α-2A/α-1A selective agonist essentially throughout at least the entire peripheral system of the subject.

Routes of peripheral administration useful in the methods of the invention encompass, without limitation, oral administration, topical administration, intravenous or other injection, and implanted minipumps or other extended release devices or formulations. An α-2A/α-1A selective agonist can be peripherally administered, without limitation, orally in any acceptable form such as in a tablet, pill, capsule, powder, liquid, suspension, emulsion or the like; an an aerosol; as a suppository; by intravenous, intraperitoneal, intramuscular, subcutaneous or parenteral injection; by transdermal diffusion or electrophoresis; topically in any acceptable form such as in drops, creams, gels or ointments; and by minipump or other implanted extended release device or formulation. An α-2A/α-1A selective agonist optionally can be packaged in unit dosage form suitable for single administration of precise dosages, or in sustained release dosage form for continuous controlled administration.

Chronic pain and other chronic conditions such as, without limitation, chronic neurological conditions can be prevented or alleviated without concomitant sedation using any of a variety of forms of repeated or continuous administration as necessary. In the methods of the invention for preventing or alleviating chronic pain or another chronic condition without concomitant sedation, means for repeated or continuous peripheral administration include, without limitation, repeated oral or topical administration, and administration via subcutaneous minipump. As non-limiting examples, a method of the invention can be practiced by continuous intravenous administration via implanted infusion minipump, or using an extended release formulation.

It is understood that slow-release formulations can be useful in the methods of the invention for preventing or alleviating chronic pain or other chronic conditions such as, without limitation, a chronic neurodegenerative conditions. It is further understood that the frequency and duration of dosing will be dependent, in part, on the prevention or extent of alleviation desired and the half-life of the selective agonist, and that a variety of routes of administration are useful for delivering slow-release formulations, as detailed hereinabove.

An α-2A/α-1A selective agonist can be peripherally administered to a subject to prevent or alleviate an ocular condition by any of a variety of means depending, in part, on the characteristics of the selective agonist to be administered and the history, risk factors and symptoms of the subject. Peripheral routes of administration suitable for preventing or alleviating an ocular condition in a method of the invention include both systemic and local administration. In particular embodiments, a pharmaceutical composition containing an α-2A/α-1A selective agonist is administered topically, or by local injection, or is released from an intraocular or periocular implant.

Systemic and local routes of administration useful in preventing or alleviating an ocular condition according to a method of the invention encompass, without limitation, oral gavage; intravenous injection; intraperitoneal injection; intramuscular injection; subcutaneous injection; transdermal diffusion and electrophoresis; topical eye drops and ointments; periocular and intraocular injection including subconjunctival injection; extended release delivery devices such as locally implanted extended release devices; and intraocular and periocular implants including bioerodible and reservoir-based implants.

In one embodiment, a method of the invention for preventing or alleviating an ocular condition is practiced by administering an ophthalmic composition containing an α-2A/α-1A selective agonist topically to the eye. The α-2A/α-1A selective agonist can be administered, for example, in an ophthalmic solution (ocular drops). In another embodiment, an ophthalmic composition containing an α-2A/α-1A selective agonist is injected directly into the eye. In a further embodiment, an ophthalmic composition containing an α-2A/α-1A selective agonist is released from an intraocular or periocular implant such as a bioerodible or reservoir-based implant.

As indicated above, an ophthalmic composition containing an α-2A/α-1A selective agonist can be administered locally via an intraocular or periocular implant, which can be, without limitation, bioerodible or reservoir-based. As used herein, the term “implant” refers to any material that does not significantly migrate from the insertion site following implantation. An implant can be biodegradable, non-biodegradable, or composed of both biodegradable and non-biodegradable materials; a non-biodegradable implant can include, if desired, a refillable reservoir. Implants useful in a method of the invention for preventing or alleviating an ocular condition include, for example, patches, particles, sheets, plaques, microcapsules and the like, and can be of any shape and size compatible with the selected site of insertion, which can be, without limitation, the posterior chamber, anterior chamber, suprachoroid or subconjunctiva of the eye. It is understood that an implant useful in the invention generally releases the implanted pharmaceutical composition at an effective dosage to the eye of the subject over an extended period of time. A variety of ocular implants and extended release formulations suitable for ocular release are well known in the art, as described, for example, in U.S. Pat. Nos. 5,869,079 and 5,443,505.

The following examples are intended to illustrate but not limit the present invention.

### Example I

### Mouse Models with Different Mechanisms of Sensory Sensitization

This example demonstrates that the increased sympathetic tone of α-2A and α-2C knockout mice enhances induction of tactile hypersensitivity by α-1 receptor activation.

A. Different Mechanisms of Sulprostone-Phenylephrine-Induced Tactile Hypersensitivity

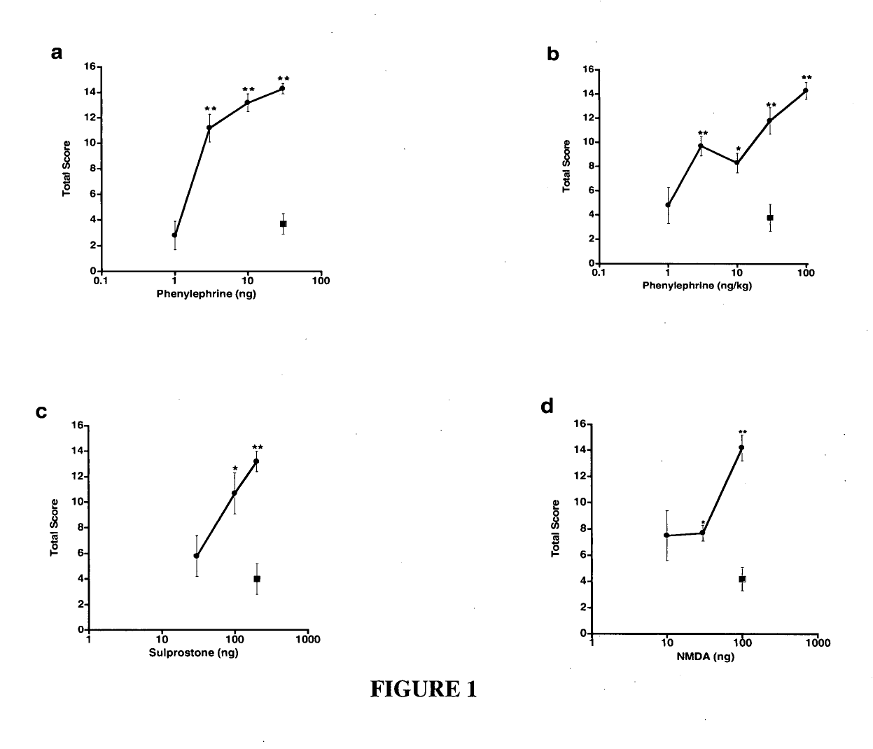

To dissect the contribution of the sympathetic nervous system to sensory sensitization, mouse models having different mechanisms of sensory sensitization were developed. Tactile hypersensitivity was measured in mice following intrathecal or intraperitoneal injection of an inducing agent by scoring the response to light stroking of the mouse flank with a paintbrush. To mimic increased sympathetic tone, phenylephrine, an α-1 adrenergic receptor agonist, was injected. As shown in FIGS. 1a and 1b, intrathecal (i.t.) or intraperitoneal (i.p.) dosing of phenylephrine caused tactile hypersensitivity, with significant responses observed starting at doses of 3 ng i.t. and 3 ng/kg i.p. Induction of tactile hypersensitivity was α-1 receptor dependent, as evidenced by the ability of the α-1 receptor antagonist 5-methyl urapidil (5-MU) to block the hypersensitive response when injected intraperitoneally.

The activity of a synthetic EP₁/EP₃ receptor-selective prostaglandin agonist, sulprostone, also was assayed. As shown in FIG. 1c, increasing doses of intrathecal sulprostone elicited dose-dependent tactile hypersensitivity; doses of 100 and 200 ng caused a significant hypersensitive response. Coadministration of a specific EP₁ receptor antagonist completely blocked the sulprostone-induced tactile hypersensitivity, demonstrating that sulprostone mediates tactile hypersensitivity through activation of the EP₁ receptor.

In a third mouse model, chemical sensitization was induced by injection of increasing intrathecal doses of NMDA, which may activate NMDA channels on post-synaptic dorsal horn neurons (Woolf et al., Science 288:1765-1769 (2000)). Intrathecal NMDA resulted in a dose-dependent tactile hypersensitivity with a maximal effect at a 100 ng dose. The hypersensitivity was blocked with the NMDA antagonist, memantine, as shown in FIG. 1d.

To assess whether the three stimuli sensitize sensory pathways by different mechanisms, a set of pharmacological agents was assayed for the ability to prevent or ameliorate tactile hypersensitivity. As shown in Table 2, each receptor antagonist (5-MU, the EP₁ receptor antagonist or memantine) blocked only tactile hypersensitivity resulting from the corresponding receptor agonist (phenylephrine, sulprostone or NMDA, respectively). Gabapentin, which is used clinically to alleviate neuropathic pain by reducing spinal sensitization, also was assayed for the ability to block tactile hypersensitivity. Gabapentin inhibited tactile hypersensitivity elicited by sulprostone and NMDA, but not by phenylephrine, further demonstrating differences between the sensory pathways involved by different stimuli.

TABLE 2
Receptor antagonists and clinically used analgesics inhibit chemically-
induced mechanical hypersensitivity

| | | | EP₁ | | |
|---|---|---|---|---|---|
| | Vehicle | 5-MU | antagonist | Memantine | Gabapentin |
| Phenyl- | 14.3 ± | 5.0 ± | 9.8 ± | 11.0 ± | 13.0 |
| ephrine | 0.7** | 1.0 | 0.7** | 0.7** | (±0.6)** |
| (100 ng/kg | | | | | |
| I.P.) | | | | | |
| Sulprostone | 13.2 ± | 12.0 ± | 4.0 ± | 14.3 ± | 3.2 ± |
| (200 ng IT) | 0.8** | 1.0** | 1.2 | 0.8** | 0.5 |
| NMDA | 14.2 ± | 13.3± | 11.4 ± | 4.2 ± | 3.7 ± |
| (100 ng IT) | 1.0** | 0.8** | 1.53* | 0.9 | 0.8 |

*indicates P < 0.01
**indicates p < 0.001

α-2 knockout mice were provided by Dr. Brian Kobilka (Stanford University; Link et al., Mol. Pharmacol. 48:48-55 (1995); Altman et al., Mol. Pharmacol. 56:154-161 (1999)). The α-2 knockout mice have a C57BL/6 background and were bred from homozygous knockout mice breeding pairs. Age and sex matched C57BL/6 wildtype mice were used as controls. Sulprostone (Cayman Chemical; Ann Arbor, Mich.) and NMDA (Sigma; St Louis, Mo.) were dissolved in dimethyl sulfoxide (DMSO). The EP₁ receptor antagonist 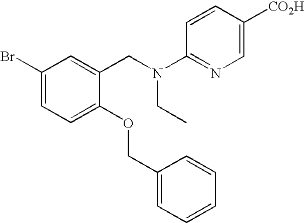
synthesized essentially as described in U.S. Pat. No. 5,843,942, and gabapentin (Victor Medical; Irvine, Calif.) were dissolved in 50% DMSO, 50% saline memantine (1-amino-3,5-dimethyladamantane hydrochloride), an analog of the well known anti-viral agent amantadine (1-adamantanamine hydrochloride), was synthesized essentially as described in U.S. Pat. No. 5,061,703 (see, also, Schneider et al., Dtsch Med. Wochenschr. 109:987 (1984)). 5-methylurapidil, brimonidine, phenylephrine, clonidine and guanethidine were obtained from Sigma and dissolved in saline. Prazosin (Sigma) and tizanidine (Biomol; Plymouth Meeting, Pa.) were dissolved in distilled water.

Spinal drug injections were performed as follows. Mice (20-30 g) were injected intrathecally as described in Hylden and Wilcox, Eur. J. Pharmacol. 67:313-316 (1980). Briefly, a sterile 30-gauge 1/2 inch needle attached to a microsyringe was inserted between the L5 and L6 vertebrae. The mouse was held firmly by the pelvic girdle in one hand, while the syringe was held in the other hand at an angle of approximately 20B above the vertebral column. The needle was inserted into the tissue to one side of the L6 spinous process, into the groove between the spinous and transverse processes. The needle angle was decreased to about 10B, and the needle slowly advanced forward into the intervertebral space until a pop was felt and there was a visible serpentine tail movement. Each compound was slowly injected in the subarachnoid space in a volume of 5 μl and was tested at multiple doses. The minimal efficacious dose was used for all subsequent experiments.

Sensitivity to light touch was quantified by scoring the response of mice to light stroking of their flanks with a small paintbrush, which is not normally painful. The mice were rated on the following scale once every 5 minutes between 15 and 50 minutes post injection: a score of “2” was given to animals showing aggressive escape responses along with squeaking and biting at the brush; a score of “1” was given to animals exhibiting mild squeaking with attempts to escape; and a score of “0” was given if the animal showed no response to the light stroking of the paintbrush. The scores were summed to generate a cumulative score of 0 to 16 as described in Minami et al., Pain 57:217-223 (1994). Statistical calculations of significance for in vivo studies were done using a two-tailed Students t-test.

B. Increased Sympathetic Tone of α-2A and α-2C Knockout Mice Enhances Their Sensitivity to Induction of Tactile Hypersensitivity by α-1 Receptor Activation

To assess whether sympathetic tone can influence susceptibility to sensory sensitization, the sensitivity of α-2A and α-2C knockout mice to chemical induction of tactile hypersensitivity was compared to the sensitivity of wildtype mice. The α-2A and α-2C knockout mice did not exhibit baseline tactile hypersensitivity when compared to wildtype controls. First, the concentration of phenylephrine that elicits tactile hypersensitivity was compared in the knockout and wildtype mice. As shown in FIG. 2, there was a dramatic leftward shift in the phenylephrine dose response in both the α-2A and α-2C knockout mice. These results demonstrate that the ability of phenylephrine to cause tactile hypersensitivity was enhanced in both α-2 knockout mouse lines, with a greater enhancement in the α-2C knockout mice. In particular, compared with a strongly tactile hypersensitivity-inducing dose of 30 ng/kg phenylephrine in the wildtype line, 0.1 and 0.3 ng/kg phenylephrine resulted in maximal hypersensitivity in the α-2C and α-2A knockout mice, respectively. As further evidenced in FIG. 2, the gradual biphasic dose-response in the wildtype mice became a steeper dose-response in both lines of knockout mice.

Systemic administration of guanethidine results in a functional sympathectomy by depleting noradrenaline from sympathetic terminals. In order to test if shifts in the phenylephrine dose response curves were due to increased sympathetic tone in the α-2 knockout mice, α-2A knockout mice were chemically sympathectomized by guanethidine treatment (50 mg/kg i.p.) and assayed for phenylephrine-induced sensitivity 24-30 hours later. In guanethidine-treated α-2A mice, the increased sensitivity to phenylephrine was partly ablated so that the dose response was similar to the biphasic dose response observed in wildtype mice (see FIG. 2). These results confirm that increased sympathetic tone enhances sensory sensitization in α-2A knockout mice.

Guanethidine sympathectomies were performed essentially as follows. Animals were injected intraperitoneally with 50 mg/kg guanethidine (Malmberg and Basbaum, Pain 76:215-222 (1998)) before being assessed for baseline tactile sensitivity 24 hours later. Animals that exhibited normal tactile sensitivity were assayed for sensitivity to chemical induction of tactile hypersensitivity. Mice recovered from the sympathectomy six to eight days later as demonstrated by a return to pre-sympathectomy responsiveness.

C. The Sympathetic Nervous System Enhances Sulprostone-Induced Tactile Hypersensitivity

Sulprostone was injected intrathecally at increasing concentrations into wildtype and α-2 knockout mice in order to determine whether the knockout mice were more sensitive to sensitization of primary afferents. As shown in FIG. 3, the dose response of sulprostone was identical in the wildtype and α-2C knockout mice, but was shifted to the left in the α-2A knockout mice. In particular, a 30 ng dose was maximally effective in the α-2A knockout mice compared to a partially hypersensitivity-inducing dose of 100 ng and a maximal dose of 200 ng in the wild-type and α-2C knockout mice. A guanethidine (50 mg/kg i.p.) chemical sympathectomy decreased the sensitivity of the α-2A knockout mice to sulprostone. As shown in FIG. 3, the dose response of sulprostone-induced tactile hypersensitivity was shifted approximately 10-fold to the right in the α-2A knockout mice treated with guanethidine. These results demonstrate that the sympathetic nervous system enhances sulprostone sensitization.

D. The Sympathetic Nervous System does not Contribute to NMDA-Induced Tactle Hypersensitivity

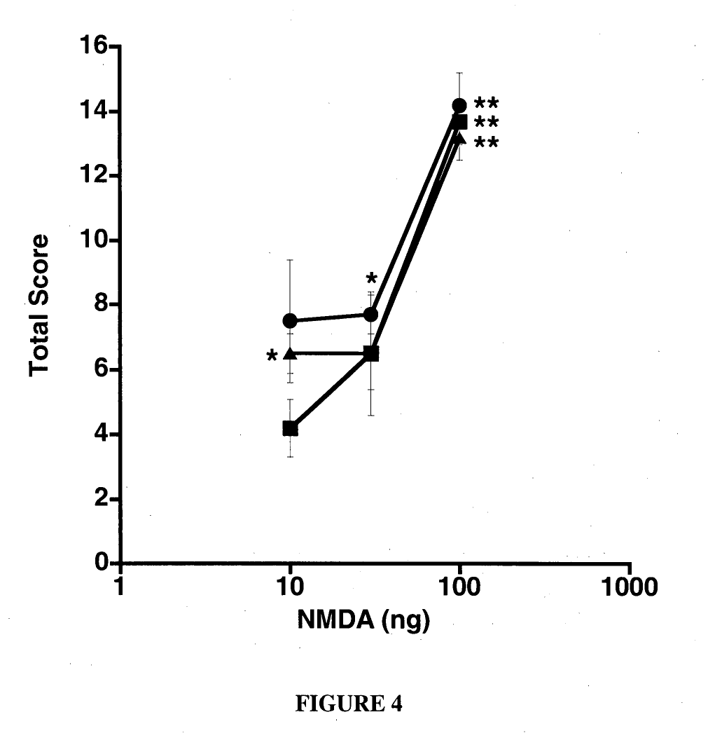

To assess whether α-2 knockout mice are more sensitive to dorsal horn sensitization by NMDA, wildtype and α-2 knockout mice were injected with varying concentrations of NMDA. As shown in FIG. 4, α-2A and α-2C knockout mice are not more sensitive to NMDA than wildtype mice. These results indicate that the sympathetic nervous system does not appear to contribute to NMDA-induced tactile hypersensitivity.

In sum, these results demonstrate that α-2 knockout mice exhibit elevated levels of sympathetic nerve activity and further indicate that these mice exhibit enhanced sensitization which is specific to the site and mode of stimulation.

### Example II

### Comparison of Activity of α-2 Agonists Brimonidine and Clonidine

This example demonstrates that α-adrenergic agonists differ in their ability to alleviate sensory hypersensitivity that is enhanced by the sympathetic nervous system.

A. Brimonidine, but not Clonidine, Alleviates Sympathetically-Enhanced Tactile Hypersensitivity

Spinally administered α-2 adrenergic agonists alleviate neuropathic pain through a spinal α-2A receptor. To determine if the increased sympathetic activity in α-2 knockout mice alters the analgesic activity of the α-2 agonists, several agonists were assayed for activity. The α-2 agonists brimonidine and clonidine were first tested in the NMDA model in which sensitization is not influenced by the basal sympathetic tone of the knockout mice. Intrathecal co-administration of NMDA with either clonidine or brimonidine resulted in complete inhibition of tactile hypersensitivity in the wildtype and α-2C (FIGS. 5a and c, respectively) knockout mice. As expected, neither clonidine nor brimonidine inhibited NMDA-induced tactile hypersensitivity in the α-2A knockout mice (FIG. 5c), consistent with previous studies showing that a spinal α-2A adrenergic receptor subtype mediates analgesic actions of α-2 adrenergic agonists (Lakhlani et al., Proc. Natl. Acad. Sci. USA 94:9950-9955 (1997); Stone et al., J. Neurosci. 17:7157-1765 (1997); Hunter et al., Br. J. Pharmacol. 122:1339-1344 (1997)). The same pattern of analgesic activity of brimonidine also was observed in the sulprostone-induced tactile hypersensitivity model, which is sensitive to sympathetic tone (see FIGS. 5b and d). In contrast, the results obtained with clonidine were strikingly different: clonidine was analgesic in wildtype mice, but not in α-2A or α-2C knockout mice (compare FIGS. 5b and d). These results demonstrate that α-2 pan-agonists can have differential activity in sympathetically-enhanced conditions, with brimonidine exhibiting activity while clonidine is inactive.

B. Brimonidine, but not Clonidine or Tizanidine, Alleviates Sulprostone-Induced Hypersensitivity in the Absence of Sedation

Sedation limits the utility of many pharmaceuticals, including α-2 agonists. The α-2 agonists were therefore compared to test whether there was a difference in the dose that resulted in alleviation of sensory hypersensitivity relative to the dose that resulted in sedation.

For three α-2 agonists (tizanidine, clonidine and brimonidine), sedative effects and the ability to block tactile hypersensitivity were compared at various doses in models of locomoter activity and sulprostone-induced tactile hypersensitivity, respectively. The tactile hypersensitivity of 5-6 mice per group was scored every five minutes between 15 and 50 minutes following intraperitoneal dosing. Vehicle treated animals typically had a score of about 4. In addition, the locomoter activity of 5-6 mice per group was measured in a five minute period 30 minutes following intraperitoneal dosing. The locomoter activity relative to vehicle-treated animals was expressed as a percentage; percentage sedation was calculated as 100% minus the percent locomoter activity. As shown in FIG. 6, of the three α-adrenergic agonists assayed, only brimonidine produced an analgesic effect that was separable from sedation. These results demonstrate that brimonidine is distinct from other α-2 pan-agonists such as clonidine and tizanidine in the ability to alleviate sympathetically-enhanced disorders such as sulprostone-induced tactile hypersensitivity without concomitant sedation.

C. Variable α-2 Versus α-1 Functional Selectivity of α-Adrenergic Pan-Agonists

The α-adrenergic receptor pharmacological profiles of brimonidine and clonidine were analyzed in assays using cell lines stably expressing α-2A, α-2C, α-1A and α-1B receptors.

Consistent with previous studies, the order of potency for inhibiting forskolin-induced cAMP accumulation in PC12 cells stably expressing either α-2A or α-2C receptor (FIGS. 7a, b; Table 3) was dexmeditomidine, which was greater than or equal to brimonidine, which was greater than clonidine, which was greater than tizanidine, which was greater than or equal to phenylephrine (Jasper et al., Biochem. Pharmacol. 55:1035-1043 (1998); Pihlavisto et al., Eur. J. Pharmacol. 385:247-253 (1999)). Brimonidine, clonidine and tizanidine were approximately 10-fold more potent at the α-2A receptor than the α-2C receptor.

The same compounds were functionally tested for the ability to stimulate α-1-mediated increases in intracellular calcium in HEK293 cells stably expressing the α-1A and α-1B receptor (FIGS. 7c, d; Table 3). The order of potency at the α-1A and α-1B receptors was phenylephrine, which was greater than clonidine, which was greater than tizanidine, which was equal to dexmeditomidine, which was greater than brimonidine. The α-2 agonists, clonidine, tizanidine and dexmeditomidine, were partial agonists while brimonidine exhibited weak activity at the α-1A receptor and no activity at the α-1B receptor. Thus, although clonidine and tizanidine have previously been characterized as “α-2 selective” agonists in binding assays, these compounds display a less than 10-fold selectivity between α-2 and α-1 receptor activation in functional assays. In contrast, dexmeditomidine was approximately 300-fold selective in functional assays, and brimonidine, the most highly selective compound in functional assays, exhibited greater than 1000-fold selectivity for α-2 receptors relative to α-1 receptors (see Table 3). These results demonstrate that brimonidine is a highly selective α-2 versus α-1 agonist and that the differential α-2/α-1 selectivity of brimonidine contrasts with the selectivity of other pan-agonists such as clonidine.

The difference in α-2/α-1 selectivity between clonidine and brimonidine indicates that the α-1 agonist activity of clonidine can augment the increased sympathetic tone of the α-2C knockout mice and mask the analgesic activity of clonidine in the sulprostone model. These results are supported by the ability of co-administration of the α-1 antagonist prazosin with clonidine to restore the analgesic activity of clonidine in α-2C knockout mice (FIG. 7e). Prazosin had no analgesic activity by itself in wildtype or α-2C knockout mice.

In sum, these results indicate that the loss of clonidine, but not brimonidine, analgesic activity in the α-2C knockout mice can be a result of clonidine's α-1 agonist activity and that the α-1 agonist activity of many “α-2 agonists” can limit their ability to treat stress-associated and other sympathetically-enhanced disorders.

Stable cell lines expressing an adrenergic receptor were established as follows. The bovine α-1A, hamster α-1B, human α-2A and human α-2C receptor cDNAs were blunt-end subcloned into the NheI-EcoRI sites in the retroviral vector pCL BABE Puro. The retroviral constructs were verified by double stranded DNA sequencing. High titer pseudotyped retroviral particles were produced by co-transfecting HEK293GP, a HEK293 cell line stably expressing Gag-Pol of the Maloney leukemia virus, with the appropriate retroviral vector and pMD.G, an expression vector for the vesicular stomatitis virus envelope protein, VSV-G. Sixteen hours after transfection, the media (DMEM, 10% FCS) was changed; the high titer (˜1×10⁶ pfu/mL) media was then harvested forty-eight hours later. The supernatant was filtered through a 0.4 uM filter.

The human α-2A and α-2C receptor supernatants were added, in varying amounts, to naive PC12 cells, which were then incubated for 48 hours. The transduced cell populations were replated at a lower density and grown in media containing 100 μg/ml puromycin. Non-transduced cells were killed within three days, and single foci grew within two months. The foci were picked, expanded, and assayed for receptor density by brimonidine radioligand binding. Functional α-2 receptor activity was confirmed by inhibition of forskolin-induced cAMP accumulation.

The bovine α-1A and hamster α-1B receptor supernatants were added, in varying amounts, to naive HEK293 cells, which were then incubated for 48 hours. The transduced cell populations were replated at a lower density and grown in media containing 0.25 μg/ml puromycin. Significant cell death was evident within three days, with single foci appearing within two weeks. After the foci were picked and expanded, expanded subclones were functionally assayed for α-1 receptor expression by measuring phenylephrine-induced intracellular Ca⁺² accumulation. Receptor density was measured in a prazosin radioligand binding assay.

Intracellular Ca⁺² responses were measured as follows in HEK293 cells stably expressing either the bovine α-1A or hamster α-1B adrenergic receptor. Between 40,000 to 50,000 cells were plated per well in 96-well poly-D-lysine coated plates in 0.2 ml DMEM containing 10% heat-inactivated fetal calf serum, 1% antibiotic-antimycotic and 0.25 μg/ml puromycin one day prior to use. Cells were washed twice with HBSS supplemented with 10 mM HEPES, 2.0 mM CaCl₂ and 2.5 mM probenicid, and subsequently incubated at 37BC for 60 minutes with 4 μM Fluo-4 (Molecular Probes; Eugene, Oreg.). The extracellular dye was washed from the plates twice prior to placing the plates in the fluorometric imaging plate reader (FLIPR; Molecular Devices; Sunnyvale, Calif.). Ligands were diluted in HBSS and aliquoted into a 96-well microplate. Drugs were tested over the concentration range of 0.64 nM to 10,000 nM. Data for Ca⁺² responses were obtained in arbitrary fluorescence units.

Intracellular cAMP measurement was performed as follows. PC12 cells stably expressing the human α-2A or human α-2C adrenergic receptors were plated in 96-well poly-D-lysine coated plates at a density of 30,000 cells per well in 100 μl DMEM supplemented with 10% horse serum, 5% heat inactivated fetal bovine serum, 1% antibiotic-antimycotic and 100 μg/ml puromycin. Cells were grown overnight at 37BC and 5% CO₂. Cells were dosed by adding an equal volume of media containing IBMX (to a final concentration of 1 mM), forskolin (to a final concentration of 10 μM) and the appropriate drug dilution (to a final concentration of between 10⁻⁵ M and 10⁻¹² M). After a 10 minute incubation, the media was aspirated and the cells lysed with 200 μl lysis buffer (Amersham Biosciences; Piscataway, N.J.). Plates were stored at −20BC for up to 24 hours prior to assay. Intracellular cAMP was determined using the Biotrak cAMP enzyme immunoassay system (Amersham Biosciences) according to the manufacturer's instructions. Plates were read on a plate reader at 450 nm.

Dose response curves for in vitro assays were generated using KaleidaGraph (Synergy Software; Reading, Pa.) by least squares fits to the equation, response=maximum response+((minimum response−maximum response)/(1+(concentration of ligand/EC₅₀)). The percent efficacy was determined by comparing the maximum effect of the compound to the effect of a standard full agonist, which was phenylephrine for α-1 receptors and brimonidine for α-2 receptors.

TABLE 3
Functional α-2 versus α-1 selectivity of α-adrenergic agonists

| | human α-2A | | human α-2C | | bovine α-1A | | hamster α-1B | | |
|---|---|---|---|---|---|---|---|---|---|
| Compound | EC50 | % E | EC50 | % E | EC50 | % E | EC50 | % E | α-1A/α-2A |
| Brimonidine | 0.86 ± 0.1 | 91 | 8 ± 3 | 93 | 1132 ± 281 | 15 | 943 ± 247 | 12 | 1316 |
| Dexmeditomidine | 0.8 ± .01 | 93 | 0.48 ± .2 | 90 | 376 ± 97 | 59 | 364 ± 72 | 62 | 289 |
| Clonidine | 10 ± 1 | 94 | 56 ± 28 | 84 | 89 ± 16 | 62 | 83 ± 10 | 63 | 8.9 |
| Tizanidine | 86 ± 35 | 93 | 1231 ± 376 | 85 | 264 ± 37 | 63 | 322 ± 31 | 61 | 3.1 |
| Phenylephrine | 306 ± 19 | 94 | 340 ± 131 | 87 | 9 ± 1 | 110 | 10 ± 1 | 110 | .03 |
| The percent efficacy (% E) was determined by comparing the maximum effect of each agonist to the maximum effect of a standard full agonist (phenylephrine for α-1 receptors and brimonidine for α-2 receptors). The values represent the mean and SEM from 3-15 independent experiments. The fold-selectivity of the agonists for α-2 receptors relative to α-1 receptors was calculated from | | | | | | | | | |
| # the ratio of their mean EC50s for activating the α-1A and α-2A receptors. | | | | | | | | | |

### Example IV

### Preparation of Compounds

This example describes preparation of several α-2 agonists.
A. Preparation of Compound 1 ((+)-(S)-4-[1-(2,3-dimethyl-phenyl)-ethyl]-1,3-dihydro-imidazole-2-thione)

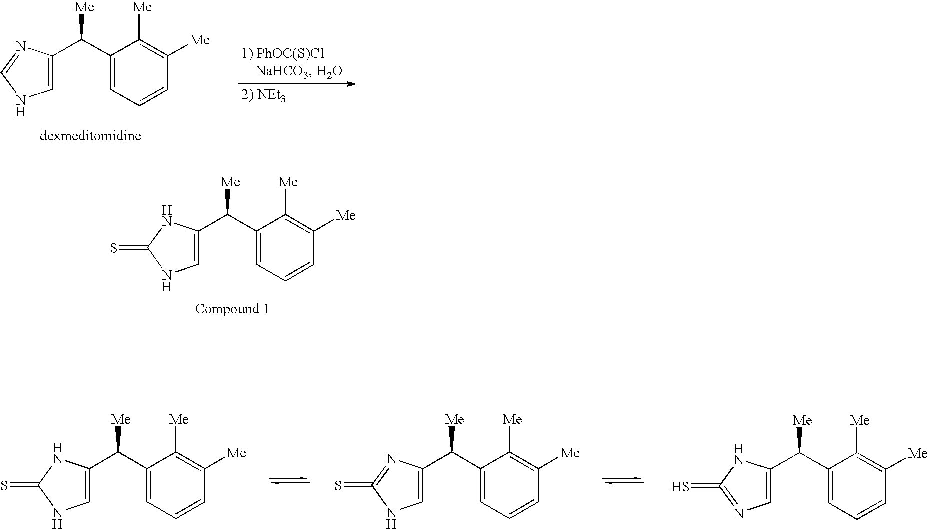

A mixture of (+)-(S)-4-[1-(2,3-dimethyl-phenyl)-ethyl]-1H-imidazole (dexmeditomidine; 2.00 g, 10.0 mmol) prepared as described in Cordi et al., Synth. Comm. 26: 1585 (1996), in THF (45 mL) and water (40 mL) was treated with NaHCO₃ (8.4 g, 100 mmol) and phenylchlorothionoformate (3.7 mL, 27.4 mmol). After stirring for four hours at room temperature, the mixture was diluted with water (30 mL) and ether (75 mL). The organic layer was removed, and the aqueous layer extracted with ether (2×50 mL). The organic layers were dried over MgSO₄ and filtered. The residue was concentrated under vacuum, diluted with MeOH (54 mL) and reacted with NEt₃ (6.5 mL) at room temperature for 16 hours. The solvent was removed under vacuum and replaced with 30% CH₂Cl₂:hexane. The solvent was removed again and solids formed. After further resuspension in 30% CH₂Cl₂:hexane, the solid was collected on a filter, washed with CH₂Cl₂:hexane and dried under vacuum to give Compound 1 ((+)-(S)-4-[1-(2,3-dimethyl-phenyl) ethyl]-1,3-dihydro-imidazole-2-thione) 1.23 g (53%).

Characterization of the product yielded the following. Optical rotation: [a]_{D}20+14° (c 1.25 in MeOH). ¹H NMR: (300 MHz, DMSO) d 11.8 (s, 1H), 11.6 (s, 1H), 7.03-7.01 (m, 2H), 6.95-6.91 (m, 1H), 6.50 (s, 1H), 4.15 (q, J=6.9 Hz, 1H), 2.25 (s, 3H), 2.20 (s, 3H), 1.38 (d, J=6.9 Hz, 3H).
B. Procedure for the Preparation of Compound 2 (5-(1H-Imidazol-4-ylmethyl)-cyclohex-1-enyl]-methanol)

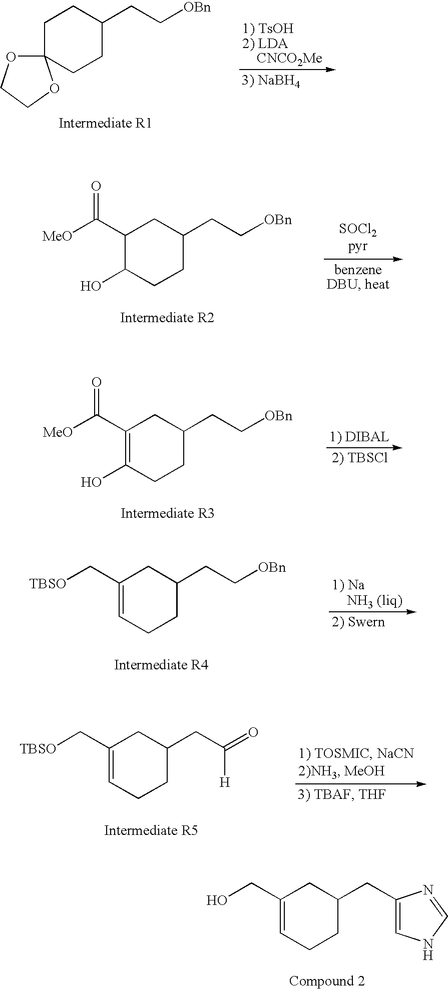

8-(2-Benzyloxy-ethyl)-1,4-dioxa-spiro[4.5]decane (Intermediate R1; 1.02 g, 3.70 mmol) was prepared as described in Ciufolini et al., J. Amer. Chem. Soc. 113: 8016 (1991). This compound was dissolved in acetone (100 mL): H₂O (5 mL) and reacted with TsOH (140 mg, 0.74 mmol) at 45EC for 5 hours. After a standard aqueous work-up the material was purified by chromatography on SiO₂ to give 4-(2-benzyloxy-ethyl)-cyclohexanone as a colorless oil (97%).

A solution of LDA (33 ml, 1.5 M in Et₂O) in THF (50 mL) at −78EC was treated with 4-(2-benzyloxy-ethyl)-cyclohexanone (9.5 g, 40.2 mmol). The mixture was warmed to 0EC over 30 minutes before re-cooling to −78EC and adding HMPA (7 mL). Methyl cyanoformate (4.1 mL, 85 mmol) was added, and the mixture stirred for 15 minutes before aqueous quench and work-up. The product was purified by chromatography on SiO₂ with 10% EtOAc:Hx. 5-(2-Benzyloxy-ethyl)-2-oxo-cyclohexanecarboxylic acid methyl ester was isolated, 5.8 g (49%), and reduced with an equivalent of NaBH₄ in MeOH at −10EC. The alcohol (Intermediate R2 above) was purified by chromatography on SiO₂ with 30 to 50% EtOAC:Hx. (˜90% yield).

A solution of 5-(2-benzyloxy-ethyl)-2-hydroxy-cyclohexanecarboxylic acid methyl ester (Intermediate R2; 0.72 g, 2.48 mmol) in pyridine (10 mL) was treated with SOCl₂ (0.73 mL, 12.4 mmol) at −20 EC. The mixture was allowed to react for 15 minutes and was then warmed to 55 EC for 16 hours. The solvents were removed under vacuum and the residue was diluted in ether at 0EC. The solution was quenched with water, washed with 1M HCl, 5% NaOH and brine. The organic material was dried over MgSO₄, filtered and freed of solvent. The mixture was diluted with benzene, and water was removed by azeotropic distillation under vacuum. The residue was dissolved in benzene (15 mL), and DBU (0.76 mL, 5 mmol) was added. The mixture was reacted for 30 minutes at room temperature. After work-up and chromatography on SiO₂ with 20% EtOAc:Hx, 5-(2-benzyloxy-ethyl)-cyclohex-1-enecarboxylic acid methyl ester (Intermediate R3) was isolated (0.56 g (82%)).

Intermediate R3 was dissolved in THF (100 mL) and added to a solution of DIBAL (70 mL, 1M in hexanes) in THF (160 mL) at −35EC for 35 minutes. The mixture was quenched with Rochelle's salt solution, and extracted with ether. The dried residue was purified by chromatography on SiO₂ with 0.30% EtOAc:Hx to yield [5-(2-benzyloxy-ethyl)-cyclohex-1-enyl]-methanol 4.6 g (80%). A solution of the alcohol (4.0 g, 18.7 mmol) in DMF (60 mL) was treated with triethylamine (3 mL) followed by TBSCl (3.0 g, 22.4 mol) for 20 minutes at room temperature. The residue was isolated from an aqueous work-up and purified by chromatography to give [5-(2-benzyloxy-ethyl)-cyclohex-1-enylmethoxy]-tert-butyl-dimethyl-silane (Intermediate R4) 3.6 g (63%).

The benzyl protected alcohol (Intermediate R4) (2.0 g, 5.55 mmol) in THF (20 mL) was cooled to −70EC, and NH₃ was condensed in this flask (˜20 mL). Na chunks were added, and the mixture was allowed to stir at −70EC for 15 minutes. The mixture was warmed to −30EC for 20 minutes, quenched with NH₄Cl, and isolated by extraction. The residue was purified by chromatography on SiO₂ with 25% EtOAc:Hx (99%). The alcohol was oxidized by the standard “Swern” protocol. The alcohol 2-[3-(tert-butyl-dimethyl-silanyloxymethyl)-cyclohex-3-enyl]-ethanol (1.3 g, 4.8 mmol) was added to a solution of oxalyl chloride (3.55 mL, 7.1 mmol) in CH₂Cl₂ (30 mL) with DMSO (0.63 mL, 8.9 mmol) at −78EC. After 40 minutes, NEt₃ (2.51 mL) was added, and the mixture was warmed to room temperature. After standard aqueous work-up and purification, [3-(tert-butyl-dimethyl-silanyloxymethyl)-cyclohex-3-enyl]-acetaldehyde (Intermediate R5) was isolated (˜95%).

The following preparation followed the procedure by Horne et al., Heterocycles 39:139 (1994). A solution of the aldehyde (Intermediate R5; 0.34 g, 1.3 mmol) in EtOH (5 mL) was treated with tosylmethyl isocyanide (TosMIC; Aldrich; 0.25 g; 1.3 mmol) and NaCN (˜15 mg, cat) and allowed to stir at room temperature for 20 minutes. The solvent was removed in vacuo; the residue was dissolved in ˜7M NH₃ in MeOH and transferred to a resealable tube before heating at 100EC for 15 hours. The mixture was concentrated and purified by chromatography on SiO₂ with 5% MeOH (sat. w/NH₃):CH₂Cl₂. A solution of the product in THF with TBAF (1.5 eq.) was stirred at room temperature after aqueous workup. The crude product was chromatographed (5-7% NH₃/MeOH in CH₂Cl₂) and designated Compound 2.

Characterization of Compound 2 yielded the following. ¹H NMR (300 MHz, DMSO-d⁶) 7.52 (s, 1H), 6.72 (s, 1H), 5.54 (brs, 1H), 3.73 (s, 2H), 2.46 (d, J=6 Hz, 2H), 1.5-2.1 (m, 6H), 1.0-1.55 (m, 1H)

### Example V

### Characterization of an α-2 Agonist with Greater α-2/α-1 Functional Selectivity than Brimonidine

This example demonstrates that α-2/α-1 selectivity in receptor proximal functional assays correlates with non-sedating in vivo activity.

A. α-2A/α-1A Functional Selectivity of Several α-2 Agonists

Proximal functional activity at the α-1A and α-2A adrenergic receptors was compared for brimonidine, dexmeditomidine, Compound 1, and Compound 2. Brimonidine was obtained from Sigma; dexmeditomidine was prepared as described in Cordi et al., supra, 1996; and Compounds 1 and 2 were synthesized as described in Example IV above. To assess α-1A activity, compounds were functionally tested for the ability to stimulate an increase in intracellular calcium in HEK293 cells stably expressing bovine α-1A receptor, described above. α-1A relative efficacy was determined in reference to the full agonist, phenylephrine, as described in Example III. As summarized in Table 4, dexmeditomidine and Compound 2 had α−1A relative efficacies greater than that of brimonidine, while the α-1A relative efficacy of Compound 1 was so low as to be undetectable in this assay.

TABLE 4
α-1a Relative Efficacy and α-1a/α-2a Potency Ratios
of Several α-2 Agonists

| | α-1A rel. | α-1A/α-2A |
|---|---|---|
| Compound | eff* | potency ratio |
| Brimonidine | 0.2 | 744 |
| Dexmeditomidine | 0.5 | 539 |
| Compound 1 | NA | — |
| Compound 2 | 0.8 | 980 |

*Efficacy relative to the reference full agonist, phenylephrine.
NA = not active

The same compounds were also functionally assayed for proximal α-2A function by assaying for inhibition of forskolin-induced cAMP accumulation in PC12 cells stably expressing human α-2A receptor. Intracellular cAMP levels were determined using the Biotrak cAMP enzyme immunoassay system described in Example III. The EC₅₀ for α-2A cAMP inhibition was expressed as a ratio with the α-1A EC₅₀ to give an α-1A/α-2A potency ratio. As shown in Table 4, Compound 2 had a higher α-1A/α-2A potency ratio than brimonidine, indicating that this compound is more selective for the α-2A receptor relative to the α-1A receptor than is brimonidine. The ratio for Compound 1 could not be determined due to the undetectable level of α-1A activity. These results indicate that Compounds 1 and 2 are highly selective for activation of α-2A as compared to α-1A. Furthermore, although dexmeditomidine has a higher α-2A potency than brimonidine (see Table 3 above), dexmeditomidine is less α-2A/α-1A selective than is brimonidine (Table 4, last column). The order of α-2A/α-1A functional selectivity of the compounds tested is Compound 2>brimonidine>dexmeditomidine. As indicated above, the α-1A/α-2A EC₅₀ ratio for Compound 1 could not be determined due to an undetectable level of α-1A activity.

B. In Vivo Efficacy and Sedative Effects

In addition to the cell-based assays described above, the various α-2 agonists were assayed for the ability to alleviate sulprostone-induced tactile hypersensitivity and sedating activity at various doses. Sulprostone-induced tactile hypersensitivity was assayed and the mean total sensitivity score calculated as described above. Locomotor activity was assayed and expressed as a percentage relative to vehicle-treated animals; percentage sedation was calculated as 100% minus the percent locomotor activity.

As shown in FIG. 8 (upper left panel), brimonidine was 60% sedating at a dose 10-fold greater than the 100 μg/kg dose which gave a 50% reduction in sulprostone sensitization. Furthermore, dexmeditomidine, shown in panel 8 (upper right panel), was completely sedating at a dose 10-fold greater than the dose required to produce a 50% reduction in sensitization score. In contrast, Compound 1, administered orally at a dose of 1 μg/kg, produced a 50% reduction in the sensitization score (solid line, left axis) with less than 30% sedation (open diamond, right axis) at doses 100-fold and even 1000-fold greater than the 1 μg/kg dose (see FIG. 8, lower left panel), and similar results were obtained with intraperitoneal administration of Compound 1. Intraperitoneal administration of Compound 2 also produced more than a 50% reduction in the sensitization score at 10 μg/kg (solid line, left axis) with less than 30% sedation at a 10-fold greater dose. Thus, Compound 1, which had an extremely low (undetectable) α-1A relative efficacy, alleviated tactile hypersensitivity without concomitant sedation upon peripheral administration. Similarly, Compound 2, which has an α-1A/α-2A potency ratio greater than that of brimonidine, also alleviated tactile hypersensitivity without concomitant sedation upon peripheral administration.

In sum, these results indicate that α-2A/α-1A adrenergic receptor functional selectivity of α-2 agonists in in vitro cell-based functional assays is associated with lack of sedative activity at the therapeutic dose following systemic or other peripheral dosing. These results further indicate that particularly useful α-2 agonists are those exhibiting α-2A/α-1A adrenergic receptor functional selectivity similar to or better than the selectivity of brimonidine.

All journal article, reference and patent citations provided above, in parentheses or otherwise, whether previously stated or not, are incorporated herein by reference in their entirety.

Although the invention has been described with reference to the examples provided above, it should be understood that various modifications can be made without departing from the spirit of the invention. Accordingly, the invention is limited only by the claims.

1. A method of preventing or alleviating a sympathetically-enhanced condition without concomitant sedation, comprising peripherally administering to a subject an effective amount of an α-2A/α-1A selective agonist, thereby preventing or alleviating said sympathetically enhanced condition without concomitant sedation,
wherein said selective agonist has an α-1A efficacy less than that of brimonidine or a ratio of α-1A/α-2A potency greater than that of brimonidine.

2. The method of claim 1, wherein said selective agonist has an α-1A efficacy less than that of brimonidine.

3. The method of claim 1, wherein said selective agonist has an α-1A/α-2A EC₅₀ ratio which is at least 30% greater than that of brimonidine.

4. The method of claim 1, wherein said effective agent has an α-1A/α-2A EC₅₀ ratio which is at least two-fold greater than that of brimonidine.

5. The method of claim 1, wherein said effective agent has an α-1A/α-2A EC₅₀ ratio which is at least ten-fold greater than that of brimonidine.

6. The method of claim 1, wherein said condition is sensory hypersensitivity.

7. The method of claim 6, wherein said condition is sensory hypersensitivity associated with headache.

8. The method of claim 7, wherein said condition is sensory hypersensitivity associated with migraine.

9. The method of claim 1, wherein said condition is gastrointestinal disease.

10. The method of claim 9, wherein said gastrointestinal disease is irritable bowel syndrome.

11. The method of claim 9, wherein said gastrointestinal disease is dyspepsia.

12. The method of claim 1, wherein said condition is a dermatological condition.

13. The method of claim 12, wherein said dermatological condition is psoriasis.

14. The method of claim 1, wherein said condition is a cardiovascular disorder.

15. The method of claim 14, wherein said condition is tachycardia.

16. The method of claim 1, wherein said condition is a disorder of peripheral vasoconstriction.

17. The method of claim 1, wherein said condition is panic attack.

18. The method of claim 1, wherein said condition is a metabolic disorder.

19. The method of claim 18, wherein said metabolic disorder is type II diabetes.

20. The method of claim 18, wherein said metabolic disorder is insulin-resistance.

21. The method of claim 18, wherein said metabolic disorder is obesity.

22. The method of claim 1, wherein said condition is a disorder of muscle contraction.

23. The method of claim 22, wherein said disorder of muscle contraction is a disorder of skeletal muscle contraction.

24. The method of claim 22, wherein said disorder of muscle contraction is a disorder of smooth muscle contraction.

25. The method of claim 22, wherein said disorder of muscle contraction is spasticity.

26. The method of claim 22, wherein said disorder of muscle contraction is associated with tension type headache.

27. The method of claim 1, wherein said condition is a behavioral disorder.

28. The method of claim 1, wherein said effective amount is administered orally.

29. The method of claim 1, wherein said effective amount is administered topically.

30. The method of claim 1, wherein said effective amount is administered via a patch.

31. A method of preventing or alleviating chronic pain without concomitant sedation by peripherally administering to a subject an effective amount of an α-2A/α-1A selective agonist, thereby preventing or alleviating said chronic pain without concomitant sedation,
wherein said selective agonist has an α-1A efficacy less than that of brimonidine or a ratio of α-1A/α-2A potency greater than that of brimonidine.

32. The method of claim 31, wherein said selective agonist has an α-1A efficacy less than that of brimonidine.

33. The method of claim 31, wherein said selective agonist has an α-1A/α-2A EC₅₀ ratio which is at least 30% greater than that of brimonidine.

34. The method of claim 31, wherein said selective agonist has an α-1A/α-2A EC₅₀ ratio which is at least two-fold greater than that of brimonidine.

35. The method of claim 31, wherein said selective agonist has an α-1A/α-2A EC₅₀ ratio which is at least ten-fold greater than that of brimonidine.

36. The method of claim 31, wherein said chronic pain is neuropathic pain.

37. The method of claim 36, wherein said neuropathic pain is associated with diabetic neuropathy.

38. The method of claim 36, wherein said neuropathic pain is associated with post-herpetic neuralgia.

39. The method of claim 31, wherein said chronic pain is associated with cancer.

40. The method of claim 31, wherein said chronic pain is post-operative pain.

41. The method of claim 41, wherein said chronic pain is allodynic pain.

42. The method of claim 41, wherein said allodynic pain is fibromyalgic pain.

43. The method of claim 31, wherein said chronic pain is associated with Complex Regional Pain Syndrome (CRPS).

44. The method of claim 31, wherein said chronic pain is visceral pain.

45. The method of claim 44, wherein said visceral pain is associated with irritable bowel syndrome.

46. The method of claim 44, wherein said visceral pain is associated with dysmennorhea.

47. The method of claim 31, wherein said chronic pain is associated with headache.

48. The method of claim 47, wherein said headache is a migraine.

49. The method of claim 47, wherein said headache is non-vascular.

50. The method of claim 47, wherein said headache is cluster headache or daily tension headache.

51. The method of claim 31, wherein said chronic pain is muscle pain.

52. The method of claim 51, wherein said muscle pain is associated with back spasm.

53. The method of claim 31, wherein said effective amount is administered orally.

54. The method of claim 31, wherein said effective amount is administered topically.

55. The method of claim 31, wherein said effective amount is administered via a patch.

56. A method of preventing or alleviating a neurological condition without concomitant sedation by peripherally administering to a subject an effective amount of an α-2A/α-1A selective agonist, thereby preventing or alleviating said neurological condition without concomitant sedation,
wherein said selective agonist has an α-1A efficacy less than that of brimonidine or a ratio of α-1A/α-2A potency greater than that of brimonidine.

57. The method of claim 56, wherein said selective agonist has an α-1A efficacy less than that of brimonidine.

58. The method of claim 56, wherein said selective agonist has an α-1A/α-2A EC₅₀ ratio which is at least 30% greater than that of brimonidine.

59. The method of claim 56, wherein said selective agonist has an α-1A/α-2A EC₅₀ ratio which is at least two-fold greater than that of brimonidine.

60. The method of claim 56, wherein said selective agonist has an α-1A/α-2A EC₅₀ ratio which is at least ten-fold greater than that of brimonidine.

61. The method of claim 56, wherein said neurological condition is an acute neurological condition.

62. The method of claim 61, wherein said acute neurological condition is stroke.

63. The method of claim 61, wherein said acute neurological condition is head or spinal cord trauma.

64. The method of claim 61, wherein said acute neurological condition is seizure.

65. The method of claim 56, wherein said neurological condition is a chronic neurological condition.

66. The method of claim 56, wherein said chronic neurological condition is a neurodegenerative disease.

67. The method of claim 66, wherein said neurodegenerative disease is Alzheimer's disease.

68. The method of claim 66, wherein said neurodegenerative disease is Parkinson's disease.

69. The method of claim 66, wherein said neurodegenerative disease is Huntington's disease.

70. The method of claim 66, wherein said neurodegenerative disease is amyotrophic lateral sclerosis or multiple sclerosis.

71. The method of claim 66, wherein said neurodegenerative disease is HIV-associated dementia or HIV-associated neuropathy.

72. The method of claim 66, wherein said neurodegenerative disease is an ocular disease.

73. The method of claim 72, wherein said ocular disease is glaucoma.

74. The method of claim 72, wherein said ocular disease is diabetic neuropathy

75. The method of claim 72, wherein said ocular disease is age-related macular degeneration.

76. The method of claim 65, wherein said chronic neurological condition is selected from schizophrenia, drug addiction, drug withdrawal, drug dependency, depression and anxiety.

77. The method of claim 56, wherein said effective amount is administered orally.

78. The method of claim 56, wherein said effective amount is administered topically.

79. The method of claim 56, wherein said effective amount is administered via a patch.

80. A method of preventing or alleviating an ocular condition without concomitant sedation by peripherally administering to a subject an effective amount of an α-2A/α-1A selective agonist, thereby preventing or alleviating said ocular condition without concomitant sedation,
wherein said selective agonist has an α-1A efficacy less than that of brimonidine or a ratio of α-1A/α-2A potency greater than that of brimonidine.

81. The method of claim 80, wherein said selective agonist has an α-1A efficacy less than that of brimonidine.

82. The method of claim 80, wherein said selective agonist has an α-1A/α-2A EC₅₀ ratio which is at least 30% greater than that of brimonidine.

83. The method of claim 80, wherein said selective agonist has an α-1A/α-2A EC₅₀ ratio which is at least two-fold greater than that of brimonidine.

84. The method of claim 80, wherein said selective agonist has an α-1A/α-2A EC₅₀ ratio which is at least ten-fold greater than that of brimonidine.

85. The method of claim 80, wherein said ocular condition is glaucoma.

86. The method of claim 80, wherein said ocular condition is macular degeneration.

87. The method of claim 80, wherein said ocular condition is retinopathy.

88. The method of claim 87, wherein said retinopathy is diabetic retinopathy.

89. The method of claim 80, wherein said effective amount is administered orally.

90. The method of claim 80, wherein said effective amount is administered topically.

91. The method of claim 80, wherein said effective amount is administered via a patch.

92. A method of screening for an α-2A/α-1A selective agonist that prevents or alleviates sympathetically-enhanced conditions without concomitant sedation upon peripheral administration, comprising determining the functional selectivity of an agent for activating an α-2A receptor as compared to an α-1A receptor,
wherein an agent which is highly selective for activating an α-2A receptor as compared to an α-1A receptor is an α-2A/α-1A selective agonist that prevents or alleviates sympathetically-enhanced conditions without concomitant sedation upon peripheral administration.

93. The method of claim 92, comprising
(a) determining potency, activity or EC₅₀ of said agent at an α-2A receptor; and
(b) determining potency, activity or EC₅₀ of said agent at an α-1A receptor,
wherein an agent which has an α-1A efficacy less than that of brimonidine or a ratio of α-1A/α-2A potency greater than that of brimonidine is an α-2A/α-1A selective agonist that prevents or alleviates a sympathetically-enhanced condition without concomitant sedation.

94. The method of claim 92, wherein said selective agonist has an α-1A efficacy less than that of brimonidine.

95. The method of claim 92, wherein said selective agonist has an α-1A/α-2A EC₅₀ ratio which is at least 30% greater than that of brimonidine.

96. The method of claim 92, wherein said selective agonist has an α-1A/α-2A EC₅₀ ratio which is at least two-fold greater than that of brimonidine.

97. The method of claim 92, wherein said effective agent has an α-1A/α-2A EC₅₀ ratio which is at least ten-fold greater than that of brimonidine.

98. The method of claim 93, wherein step (a) comprises assaying for inhibition of adenylate cyclase activity.

99. The method of claim 98, wherein said assaying for inhibition of adenylate cyclase activity is in PC12 cells stably expressing α-2A.

100. The method of claim 99, said PC12 cells stably expressing human α-2A.

101. The method of claim 93, wherein step (b) comprises assaying for intracellular calcium.

102. The method of claim 101, wherein intracellular calcium is assayed in HEK293 cells stably expressing α-1A.

103. The method of claim 102, said HEK293 cells stably expressing bovine α-1A.
